(19) United States
(12) Patent Application Publication
(10) Pub. No.: US 2002/0123134 A1
(43) Pub. Date: Sep. 5, 2002

(54) Active and biocompatible platforms prepared by polymerization of surface coating films

(76) Inventors: Mingxian Huang, San Diego, CA (US); Xiaobo Wang, San Diego, CA (US); Lei Wu, San Diego, CA (US); Weiping Yang, San Diego, CA (US); Jing Cheng, Beijing (CN)

Correspondence Address: DAVID R PRESTON & ASSOCIATES, 12625 HIGH BLUFF DRIVE, SUITE 205, SAN DIEGO, CA, 92130, US

(21) Appl. No.: 10/022,058

(22) Filed: Dec. 13, 2001

Related U.S. Application Data
(60) Provisional application No. 60/258,281, filed on Dec. 26, 2000.

(51) Int. Cl. C12M 1/34; C12Q 1/68; G01N 33/53; G01N 33/542

(52) U.S. Cl. 435/287.2; 435/6; 435/7.9

(57) ABSTRACT

The present invention recognizes that polymerizable coating films can be utilized to make chips such as biochips that include channel structures. These chips can optionally include one or more additional structures such as particles, biological groups or chemical groups. Such biochips having channel structures have a wide variety of useful applications, particularly in the field of laboratory on a chip and other applications where microfluidics are of importance. One aspect of the present invention is a platform that includes: a surface, a coating film and a channel structure. Preferably, the coating film defines in part said channel structure and more preferably the platform comprises a microchip.

This patent application claims benefit of priority to United States provisional patent application No. 60/258,281, filed Dec. 26, 2000, naming Huang et al. as inventors, which is incorporated by reference in its entirety herein.

The following applications are incorporated herein by reference in their entirety:

U.S. application Ser. No. 09/636,104 filed on Aug. 10, 2000, entitled “Method for Manipulating Moieties in Microfluidic Systems” naming as inventors Wang et al.;

PCT Application Number PCT/US99/21417 filed on Sep. 17, 2999, entitled “Individually Addressable Micro-Electromagnetic Unit Array Chips;”

U.S. patent application Ser. No. 09/679,024 filed on Oct. 4, 2000 entitled “Apparatuses Containing Multiple Active Force Generating Elements and Uses Thereof” and naming as inventors Wang et al., which corresponds to People's Republic of China Application Number (to be determined) and having attorney docket number I2000725EB, filed Sep. 30, 2000;

U.S. application Ser. No. 09/686,737 filed Oct. 10, 2000, entitled “Compositions and Methods for Separation of Moieties on Chips” that corresponds to People's Republic of China Application No. (to be determined) having attorney docket number I2000726EB filed Oct. 9, 2000;

U.S. applications Ser. No. 60/239,299 filed Oct. 10, 2000, entitled “An Integrated Biochip System for Sample Preparation and Analysis;”

U.S. application Ser. No. 09/685,410 filed Oct. 10, 2000, entitled “Individually Addressable Micro-Electromagnetic Unit Array Chips in Horizontal Configuration;”

U.S. patent application Ser. No. 09/678,263 having filed on Oct. 3, 2000, entitled “Apparatus for Switching and Manipulating Particles and Method of Use Thereof” and naming as inventors Wang et al., which corresponds to U.S. application Ser. No. 09/678,263 entitled “Apparatus for Switching and Manipulating Particles And Methods Of Use Thereof” filed Sep. 27, 2000;

U.S. application Ser. No. 09/684,081 filed Aug. 25, 2000, entitled “Methods and Compositions for Identifying Nucleic Acid Molecules Using Nucleolytic Activities and Hybridization;”

U.S. application Ser. No. 09/636,104 filed Aug. 10, 2000, entitled “Methods for Manipulating Moieties in Microfluidic Systems;”

U.S. application Ser. No. 09/399,299 filed Sep. 17, 1999, entitled “Individually Addressable Micro-Electromagnetic Unit Array Chips.”

### TECHNICAL FIELD

The present application concerns micro-devices known as “biochips” and more particularly methods of making biochips using selective polymerization of coating films and methods of using such biochips.

### BACKGROUND

As a novel and emerging technology in life science and biomedical research during last several years, biochip technology can be applied to many areas of biology, biotechnology and biomedicine including point-mutation detection, DNA sequencing, gene expression, drug screening and clinical diagnosis. Biochips refer to miniaturized devices that can be used for performing chemical reactions, biochemical reactions, detection of such reactions and sample separations. Biochips are produced using microelectronic and microfabrication techniques as used in semiconductor industry or other similar techniques, and can be used to integrate and shrink the currently discrete chemical or biochemical analytical processes and devices into microchip-based apparatus. Recent scientific literature shows a plethora of uses for these devices. The reader's attention is drawn to the following articles for an appreciation of the breadth of biochip uses. “Rapid determination of single base mismatch mutations in DNA hybrids by direct electric field control” by Sosnowski, R. G. et al. (Proc. Natl. Acad. Sci., USA, 94:1119-1123 (1997)) and “Large-scale identification, mapping and genotyping of single-nucleotide polymorphisms in the human genome” by Wang, D. G. et al. (Science, 280: 1077-1082 (1998)) show current biochip use in detection of point mutations. “Accurate sequencing by hybridization for DNA diagnostics and individual genomics” by Drmanac, S. et al. (Nature Biotechnol. 16: 54-58 (1998)), “Quantitative phenotypic analysis of yeast deletion mutants using a highly parallel molecular bar-coding strategy” by Shoemaker, D. D. et al. (Nature Genet., 14:450-456 (1996)), and “Accessing genetic information with high density DNA arrays.” by Chee, M et al., (Science, 274:610-614 (1996)) show biochip technology used for DNA sequencing. The use of biochip technology to monitor gene expression is shown in “Genome-wide expression monitoring in Saccharomyces cerevisiae” by Wodicka, L. et al (Nature Biotechnol. 15:1359-1367 (1997)), “Genomics and human disease-variations on variation.” by Brown, P. O. and Hartwell, L. and “Towards Arabidopsis genome analysis: monitoring expression profiles of 1400 genes using cDNA microarrays” by Ruan, Y. et al. (The Plant Journal 15:821-833 (1998)). The use of biochips in drug screening is illustrated in “Selecting effective antisense reagents on combinatorial oligonucleotide arrays” by Milner, N. et al. (Nature Biotechnol., 15:537-541 (1997)), and “Drug target validation and identification of secondary drug target effects using DNA microarray” by Marton, M. J. et al. (Nature Medicine, 4:1293-1301 (1998)). Examples of clinical diagnostic use of biochips is illustrated in “Cystic fibrosis mutation detection by hybridization to light-generated DNA probe arrays” by Cronin, M. T. et al. (Human Mutation, 7:244-255 (1996)), and “Polypyrrole DNA chip on a silicon device: Example of hepatitis C virus genotyping” by Livache, T. et al. (Anal. Biochem. 255:188-194 (1998)). These references are intended to give a notion of the wide range of DNA biochip uses.

A variety of biochips have biomolecules (for example, oligonucleotides, cDNA and antibodies) immobilized on their surfaces. There are a number of different approaches to make such chips. For example, the light-directed chemical synthesis process developed by Affymetrix (for example, U.S. Pat. Nos. 5,445,934 and 5,856,174) is a method of synthesizing biomolecules on chip surfaces by combining solid-phase photochemical synthesis with photolithographic fabrication techniques. The chemical deposition approach developed by Incyte Pharmaceutical uses pre-synthesized cDNA probe for directed deposition onto chip surfaces (see, for example, U.S. Pat. No. 5,874,554). The contact-print method developed by Stanford University uses high-speed, high-precision robot arms to move and control liquid-dispense head for directed cDNA deposition and printing onto chip surfaces (see, for example, Schena, M. et al. Science 270:467-70 (1995)). The University of Washington at Seattle developed a single-nucleotide probe synthesis method by using four piezoelectric deposition heads, which are loaded separately with four types of nucleotide molecules to achieve required deposition of nucleotides and simultaneous synthesis on chip surfaces (see for example, Blanchard, A. P. et al. Biosensors & Bioelectronics 11:687-90 (1996)). Hyseq, Inc. has developed passive membrane devices for sequencing genomes (see, for example, U.S. Pat. No. 5,202,231).

There are two basic types of biochips, for example, passive and active. Passive biochips refer to those on which chemical or biochemical reactions are dependent on passive diffusion of sample molecules. In active biochips reactants are actively moved or concentrated by externally applied forces so that reactions are dependant not only on simple diffusion but also on the applied forces. The majority of the available biochips, for example, oligonucleotide-based DNA chips from Affymetrix and cDNA-based biochips from Incyte Pharmaceuticals, belong to the passive type. There are structural similarities between active and passive biochips. Both types of biochips employ of arrays of different immobilized ligands or ligand molecules. By using various markers, detectable markers, detection systems and indicator molecules (for example, fluorescent dye molecules), the reaction between ligands and other molecules can be monitored and quantified. Thus, an array of different ligands immobilized on a biochip allows for the reaction and monitoring of multiple analyte molecules.

Many current passive biochip designs do not take full advantage of microfabrication and microelectronic technologies. Passive biochips cannot be readily used to achieve fully integration and miniaturization of the entire bioanalytical system from the front-end sample preparation to final molecular quantification/detection. In addition, passive biochips have other disadvantages including low analytical sensitivity, a long reaction time, and difficulties associated with control of temperature, pressure, and electrical fields at individual sites (called units) on the chip surfaces as well as difficulties in controlling the local concentrations of molecules.

On the other hand, active biochips allow versatile functions of molecular manipulation, interaction, hybridization reaction and separation (such as PCR and capillary electrophoresis) by external forces through means such as microfluidic manipulation and electrical manipulation of molecules. However, many such biochips cannot be readily used in high throughput applications. The electronic biochips developed by Nanogen can manipulate and control sample biomolecules with electrical field generated by microelectrodes, leading to significant improvement in reaction speed and detection sensitivity over passive biochips (see, for example, U.S. Pat. Nos. 5,605,662, 5,632,957, and 5,849,486). However, to effectively move biomolecules in their suspension/solutions with electrical fields, electrical conductivity of solutions has to be very low. This significantly limits the choice of buffer solutions used for biochemical assays. Many enzymes and other biomolecules are denatured under conditions of low ionic strength and/or serious non-specific binding occurs to chip surfaces. Multi-force chips can overcome these types of problems, particularly chips that include magnetic elements, because magnetic forces tend not to be limited by the type of suspending media being used, such as the type and character of buffer being used.

Microchips have gained recognition in the field of miniaturized high-throughput analysis of samples such as biological samples. The fabrication of microchips has progressed in two fields, injection molding and machining. For example, poly(dimethylsiloxane) (PDMS) has been used to fabricate microcontact stamps and microfluidic channels. Injection-molded plastic substrates have also been used to make microchannel separation devices. Microseparation channels in plastic substrates have also been made using laser ablation. These approaches have tended to facilitate the fabrication of microchip devices on substrates other than glass or silica. However, plastic materials that are used in microchip manufacture are hydrophobic and are not particularly biocompatible in nature. Also, the contact surfaces made during these processes tend to require additional modification so that they are appropriate for biological assays. However, surface modification methods are limited and therefore limit the application and capabilities of the biochip. The present invention addresses these and other shortcomings in these methods and biochips.

The present invention utilizes polymerizable coating films, preferably coating films that are designed to be active and biocompatible. Particles may be imbedded within the coating film such that regions of interest are exposed on the surface. The coating film can be selectively polymerized, such as by the use of polymerizing initiators that are responsive to heat or light. The use of appropriate masking leads to selective polymerization in certain areas and reduced or non-polymerization in other areas. The reduced or non-polymerized materials are removed, such as through washing, such that channel structures are formed. Alternatively, a particle imbedded platform may be molded by baking or by photopolymerizing a mixture of polymers and particles introduced into a substrate having a desired structure..

### BRIEF DESCRIPTION OF THE FIGURES

FIG. 1 depicts one aspect of a biocompatible platform 10 where a platform 11 includes a surface 12 upon which is a coating film 13 that includes channel structures 14 that define an island 15. In one aspect of the present invention, the platform and the surface are the same.

FIG. 2a and 2b generally depict the particle in its general location within the present invention and a representation of possible chemical and biological groups that may be in contact with the particle. More specifically, FIG. 2a depicts an open channel structure defined by a coating film 13 that includes particles 16. FIG. 2b is a representation of the different biological and chemical groups a particle may have. More specifically the particle may have biological groups such as but not limited to a nucleic acid molecule 17a, a specific binding moiety 17b, or a peptide 17c. The chemical groups may be positively charged 18a, negatively charged 18b, or have no charge 18c, and may be hydrophilic or hydrophobic. Biological and chemical moieties may be in displayed either alone or in combination.

FIG. 3 depicts a platform 10 having a platform 11 that includes channel structures 14 that define an island 15 and additional structures, such as acoustic elements 19, electron magnetic elements 20, magnetic elements 21 and dielectric elements 22. These additional structures are depicted as being within the surface 12, below the coating film 13 and oriented to be below channel structures 14. These additional structures can be used in assays to move or modulate materials, including particles such as cells.

FIG. 4 depicts a plurality of layers that may form a platform either independently or in conjunction with one another that includes a coating film 13 having a channel 14 and particles 16, an acoustic element 19, an electromagnetic element 20, a dielectric element 22, a cover 40, and can be provided in a single or multiple layers.

FIG. 5a through 5c depict a variety of channel and chamber configurations of the present invention. More specifically, FIG. 5a depicts a sample reservoir 28, a sample channel 29, a separation channel 26, and a detection portion 27. FIG. 5b refers to a configuration having a sample introduction site 25, a separation channel 26, and a detection portion 27. FIG. 5c refers to a configuration having a sample introduction site 25, a reaction well 24, a separation channel 26, and a detection portion 27.

FIG. 6 depicts a silicon wafer platform 10 having about a four inch diameter including a variety of channel and chamber configurations of the present invention.

FIG. 7 depicts a silicon wafer platform 10 having about a four inch diameter including a variety of channel and chamber configurations of the present invention.

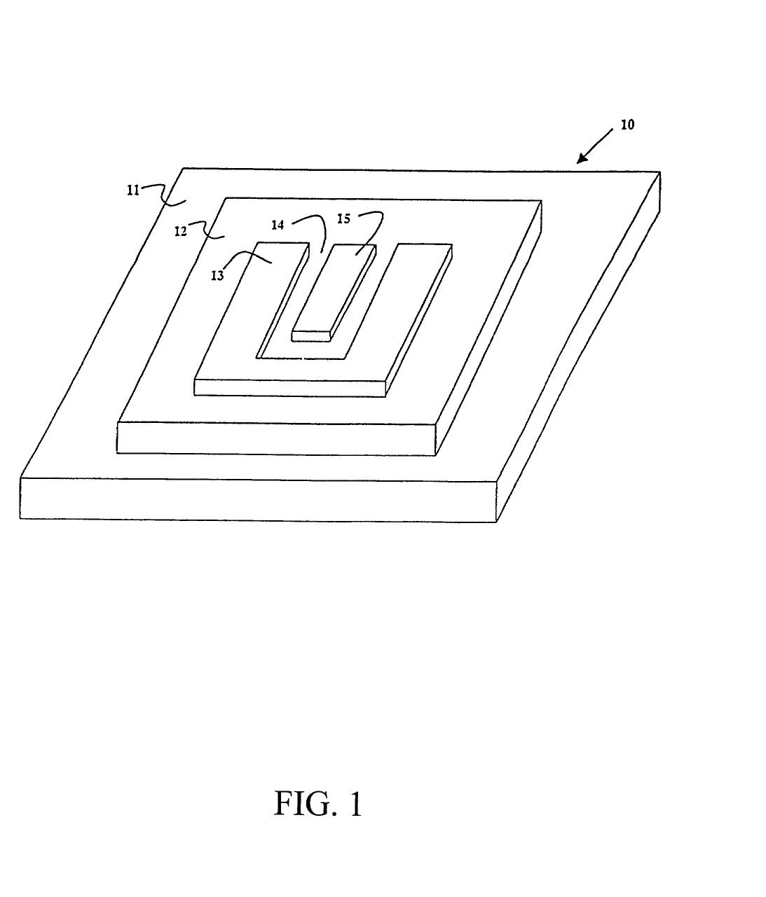

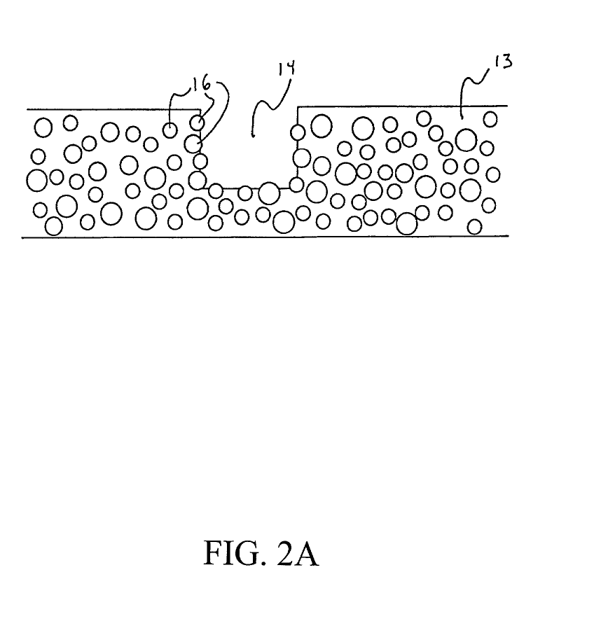

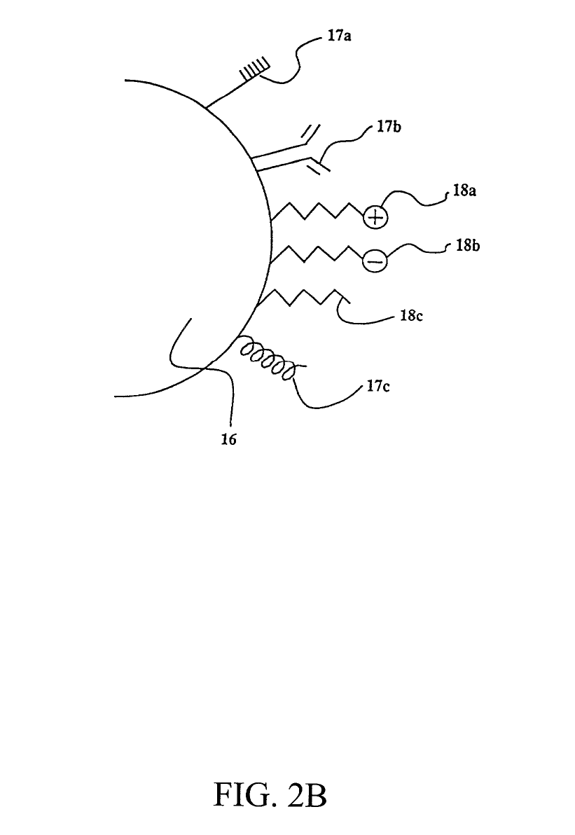

### SUMMARY

The present invention recognizes that polymerizable coating films can be utilized to make chips such as biochips that include channel structures. These chips can optionally include one or more additional structures such as particles, biological groups or chemical groups. Such biochips having channel structures have a wide variety of useful applications, particularly in the field of laboratory on a chip and other applications where microfluidics are of importance.

A first aspect of the present invention is a platform that includes: a surface, a coating film and a channel structure. Preferably, the coating film defines in part said channel structure and more preferably the platform comprises a microchip. The surface can be any appropriate surface, but is preferably made of silica, glass, quartz or fused silica. The coating film can be of any appropriate materials, such as polymers, such as homopolymers, copolymers, hydrophilic polymers or hydrophobic polymers. The coating film can include a biological group that can, for instance, interact with a biological moiety, such as through specific binding reactions such as, but not limited to, protein-protein, nucleic acid—nucleic acid or protein—nucleic acid interactions. The coating film can also include chemical groups that can interact with biological moieties or chemical moieties through chemical interactions, such as through chemical reactions, such as those that form covalent bonds or non-covalent bonds. The coating film preferably includes particles of appropriate materials, such as glass, silica, quartz or plastics. The particles can strengthen the coating film, such as to allow for the coating film to be of increased thickness, which can allow for channel structures to be of greater strength and depth than if the particles were not present. The particles, like the polymer component of the coating film, can also include biological groups or chemical groups, or combinations thereof. The channel structures are formed at least in part by the coating film. The channel structures are preferably formed using selective photopolymerization of the coating film, such as through masking, such that unpolymerized regions of polymerizable material during manufacture can be removed, such as by washing, to form the channels. The channel structures can include biological groups or chemical groups, or combinations thereof. The biological groups and/or chemical groups can be provided on the coating film or on particles. In one aspect of the present invention, the biological groups and/or chemical groups are present substantially throughout the coating film. In these and other configurations, it is preferable such that the biological groups or chemical groups are exposed on the surface of the chip, preferably on at least one surface of a channel. When moieties such as chemical moieties or biological moieties pass along a channel, the movement of such moieties can be modified due to forces acting upon the moieties by the chemical groups or biological groups. In one preferred aspect of the present invention, the platform can include a variety of additional structures, preferably those that can modulate the movement of particles or biological entities on the chip, such as through the channel structures. Preferred additional structures include, but are not limited magnetic elements, electromagnetic elements, acoustic elements or dielectric elements.

A second aspect of the present invention is a method of making a platform that includes at least one channel structure. The method includes: providing a surface and contacting the surface with a polymerizable composition. The polymerizable composition preferably includes: unpolymerized polymer subunits and at least one polymerization initiator. The polymerizable composition is preferably selectively polymerized at loci to form a platform that includes a polymerized layer that defines at least in part at least one channel structure. The surface can be any appropriate surface, such as glass, quartz or plastic. The polymerizable composition can include appropriate monomers, macromonomers or combinations thereof. The polymerizable composition can be polymerized to form a homopolymer, copolymer, cross-linked polymer or a polymer network. Preferably, the unpolymerized polymer subunits and the polymerized product are biocompatible, but that is not a requirement of the present invention. Polymerization of the polymerizable composition is preferably initiated with an appropriate initiator, such as a photoinitiator, thermal initiator or a combination thereof. Polymerization of the polymerizable composition can be selective by use of masks, such as masks appropriate for use in combination with photoinitiators or thermal initiators or combinations thereof. Preferably, the initiator is a photoinitiator that used in combination with a mask and a highly localized and focused source of light, such as a laser, to initiate polymerization at loci, preferably predetermined loci. The polymerizable composition can also optionally include a chemical group, a biological group or a combination thereof. In one aspect of the present invention, these groups become trapped and/or bonded in the polymerized product, preferably where at least a portion of functional groups associated with such biological groups or chemical groups are exposed to channel structures. The polymerizable composition can also include a polymerizing functional group, preferably associated with a monomer, macromonomer, polymer or partially polymerized polymer such that a cross-linked polymer matrix can result, but that is not a requirement of the present invention. The polymerizable composition can also include particles that optionally include biological groups, chemical groups or combinations thereof. Preferably, such biological groups or chemical groups are exposed to a channel structure upon polymerization. Channel structures are preferably formed after polymerization, such as selective polymerization. Unpolymerized material is removed, such as through washing, so that channel structures are formed. The surface or platform can include a variety of additional structures, preferably those that can modulate the movement of particles or biological entities on the chip, such as through the channel structures. Preferred additional structures include, but are not limited magnetic elements, electromagnetic elements, acoustic elements or dielectric elements. The present invention also includes a platform, chip or biochip made by a method of the present invention.

A third aspect of the present invention is a method of separating moieties such as biological and chemical moieties that includes: providing a platform of the present invention, providing a sample containing moieties, contacting the platform with the sample, moving the sample through channels on the platform such that moieties within the sample are separated and optionally detecting at least one moiety. In one aspect of the present invention, the method: providing a platform of the present invention, providing a sample containing moieties, contacting the platform with a sample, moving the sample through channels on the platform such that moieties within the sample are separated and optionally manipulated and optionally detecting at least one moiety. The separation methods can include high performance liquid chromatography (HPLC), capillary electrophoresis (CE), and capillary electrochromatography (CEC) using the channels on the platform. The manipulation of sample moieties is preferably performed by applying appropriate external forces through means such as microfluidic devices or by applying appropriate electric or magnetic forces.

A fourth aspect of the present invention is a method of performing a chemical reaction, biochemical reaction or a bioassay that includes: providing a platform of the present invention, providing one or more reagents for use in the bioassay, contacting the platform with the reagents, moving the reagents through channels on the platform such that the reagents are contacted and a bioassay is performed and optionally detecting at least one reactant or product of the bioassay. In one aspect of the present invention includes a method for performing chemical reactions or biochemical reactions that includes: providing a platform of the present invention, providing one or more reagents for use in the reactions, contacting the platform with the reagents, moving and optionally mixing the reagents through channels on the platform such that reactions can occur and optionally detecting the occurrence of a chemical reaction or a biochemical reaction.

A fifth aspect of the present invention is a method for cell separation or cell capture. In one aspect of the present invention a method for cell separation is disclosed that includes: providing a platform of the present invention, injecting or introducing a sample having cells into at least one channel structure on the platform, moving the sample or at least one component thereof through at least one channel structure on the platform such that the cells within the sample are separated, and optionally detecting the cells. In another aspect of the present invention a method for cell capture is disclosed that includes: providing a platform of the present invention, introducing a sample having cells into at least one channel structure on the platform, moving the sample or at least one component thereof through at least one channel structure on the platform such that the cells within the sample are captured, and optionally detecting the cells.

### DETAILED DESCRIPTION OF THE INVENTION

Definition

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Terms of orientation such as “up” and “down” or “upper” or “lower” and the like refer to orientation of parts during use of a device. Where a term is provided in the singular, the inventors also contemplate the plural of that term. The nomenclature used herein and the laboratory procedures described below are those well known and commonly employed in the art. Where there are discrepancies in terms and definitions used in references that are incorporated by reference, the terms used in this application shall have the definitions given herein. As employed throughout the disclosure, the following terms, unless otherwise indicated, shall be understood to have the following meanings:

“Platform” refers to a structure that includes at least one microchip structure, including a biochip structure.

“Surface” refers to a portion of a platform that is in contact with a coating film.

“Microchip” refers to a miniaturized structure that includes channel structures, wells or chambers.

“Biochip” refers to a microchip that is capable of performing biological reactions, chemical reactions, detections or analyses.

“Coating film” refers to a film coating, such as a coating including polymers and optionally particles. The coating film is preferably of a defined thickness and a defined composition.

“Channel structure” refers to structures on a microchip that are open or closed channels capable of holding fluids, gasses or solids. Open channels are channels that are open on at least one side in cross-section, such as by analogy a channel used in waterways. Closed channels are channels that are closed on all sides in cross-section, such as by analogy a pipe used to transfer water underground. Channel structures can also form wells or chambers. “Well” refers to structures on a microchip that can act as a reservoir or mixing chamber that can hold a sample, fluid, reagent or liquid. “Chamber” refers to structures on a microchip that can act as a reservoir or mixing chamber that can hold a sample, fluid, reagent or liquid. Wells are open structures whereas chambers are closed structures. For example, wells are open to the surface of a chip, but can have channel structures present that allow materials to flow into or out of the wells. Chambers, on the other hand, are covered by a covering structure for form a closed structure and can have channel structures present that allow materials to flow into or out of the chambers. In one aspect of the present invention, chambers or wells are connected by channel structures

“Magnetic element” refers to a structure under, within or on a platform, surface, microchip or biochip capable of creating a magnetic field, such as a magnetic field that exerts a force in, on or near a platform, surface, microchip or biochip. Magnetic elements include electromagnetic elements.

“Electromagnetic element” refers to a structure under, within or on a platform, surface, microchip or biochip capable of creating a magnetic field by electromagnetism, such as a magnetic field that exerts a force in, on or near a platform, surface, microchip or biochip.

“Acoustic element” refers to a structure under, within or on a platform, surface, microchip or biochip capable of creating sound wave, such as those that can aid in the forming of currents, such as to aid mixing, in a fluid or solid, such as acoustics that exert a force in, on or near a platform, surface, microchip or biochip.

“Dielectric element” refers to a structure under, within or on a platform, surface, microchip or biochip capable of creating a dielectric field, such as a dielectric field in, on or near a platform, surface, microchip or biochip.

“Capillary electrophoresis” or “CE” refers to a technique, preferably an analytical technique, that separates species by applying high voltage across buffer filled small inner diameter capillaries. It is generally used for separating charged species, which move at different speeds when the voltage is applied depending on their size and charge. The solutes are seen as peaks as they pass through the detector and the area of each peak is proportional to their concentration. The capillary can be filled by polymer gel solution or reversed micellar solution to enhance the separation.

“High performance liquid chromatography” or “HPLC” refers to a method that uses a liquid mobile phase to separate the components of a mixture. These components (or analytes) are first dissolved in a solvent, and then forced to flow through a chromatographic column under a high pressure. In the column, the mixture is resolved into its components. The amount of resolution is important, and is dependent upon the extent of interaction between the solute components and the stationary phase. The stationary phase is defined as the immobile packing material in the column which allows separations based on reversed phase, size exclusion, ion exchange, and affinity interaction. The interaction of the solute with mobile and stationary phases can be manipulated through different choices of both solvents and stationary phases. As a result, HPLC acquires a high degree of versatility not found in other chromatographic systems and it has the ability to easily separate a wide variety of chemical mixtures.

“Capillary electrochromatography” or “CEC” refers to a hybrid technique between HPLC and CE. In essence, CE capillaries are packed with HPLC packing and a voltage is applied across the packed capillary that generates an electro-osmotic flow (EOF). The EOF transports solutes along the capillary towards the detector. Both differential partitioning and electrophoretic migration of the solutes occurs during their transportation towards the detector which leads to CEC separations.

“Photomask” refers to high precision plates containing microscopic images of microchannels. Photomasks are made from very flat pieces of quartz or glass with a layer of chrome on one side. In photolithography, photoresist coated on the wafer can be broken down or polymerized upon projecting the image on the photomask onto the wafer. The wafer is then developed and etched to form desired microstructure.

“Biocompatable” refers to a characteristic of a composition of matter, compound, material or structure to be substantially compatible with biological groups, biomolecules or biological moieties. To be substantially compatible refers to properties that include non-denaturing, not substantially altering or diminishing biological activity, not substantially adversely affecting bioreactions, biospearations or biological processes such as, but not limited to enzyme activity, specific binding reactions, cellular activity, cellular motility or the like.

“Biological group” refers to a moiety of biological origin, of biological character or small molecules, such as but not limited to nucleic acid molecules (DNA, RNA, single stranded, double stranded, triple stranded or combinations thereof), proteins or polypeptides, lipids, carbohydrates or combinations thereof. Biological groups are capable of interacting with a biological moiety, chemical group, chemical moiety or small molecule by non-covalent interactions. For example, a biological group can include an antibody that interacts with an antigen or small molecule, a nucleic acid molecule that interacts with another nucleic acid molecule or a polypeptide, or a receptor that interacts with a ligand or chemical group. A biological group is preferably immobilized (directly, indirectly, reversibly, non-reversibly or imbedded) to a surface but that is not a requirement of the present invention.

“Biological moiety” refers to a moiety of biological origin, of biological character or a small molecule that is capable of interacting with a biological group, chemical group, chemical moiety or small molecule by non-covalent interactions. For example, a biological moiety can be an antigen or small molecule that interacts with an antibody, a nucleic acid molecule that interacts with another nucleic acid molecule or polypeptide or a ligand or chemical moiety that interacts with a receptor. A biological moiety may be a cell, a population of cells, or may be an etiological agent. Biological moieties may be found in homogenous solutions such as, but not limited to, cloned cell cultures or may be found in heterogeneous mixtures such as, but not limited to, blood, maternal blood, or bodily fluid or tissue. A biological moiety is preferably present in a sample that can interact with an immobilized biological group.

“Chemical group” refers to moiety of inorganic chemical, organic chemical, biological origin, biological character or a small molecule that can interact with a chemical moiety, biological group, biological moiety or small molecule to form a covalent bond or a chemical reaction. For example, a chemical group can include functional groups that can interact with a biological group to form a covalent bond. A chemical group is preferably immobilized (directly, indirectly, reversibly, non-reversibly or imbedded) to a surface but that is not a requirement of the present invention.

“Chemical moiety” refers to a moiety of inorganic chemical, organic chemical, biological origin, biological character or a small molecule that can interact with a chemical group, biological group, biological moiety or small molecule to form a covalent bond or a chemical reaction. A biological moiety is preferably present in a sample that can interact with an immobilized biological group.

“Moiety” refers to any moiety including for example a biological moiety and/or a chemical moiety. A moiety may be a portion of a moiety or may be the entire moiety.

“Small molecule” refers to an inorganic or organic molecule or biological or chemical origin that has or is suspected of having at least one bioactivity. Small molecules include, for example, antibiotics, ions, sugars, carbohydrates, fatty acids, nucleotides, prostoglanding, drugs or compounds suspected of having activities of drugs and the like.

“Particle” refers to a particulate of any shape or size that is appropriate to be provided in a coating film or a polymerizable composition to form a coating film. Particles in a coating film preferably add support to the coating film so that the coating film can support channel structures, particular channel structures of greater depth or proximity to other channel structures than if the particles were not present in the coating film. Particles can also include biological groups or chemical groups, but this is not a requirement of the present invention. Particles can also optionally include additional structures such as pores, such as provided on dextran particles, such as Sephadex™. Particles can also optionally be light emitting particles, such as Quantum Dots as they are known in the art (see, for example, Springholz et al., Science 5389:734-737 (1998); Ross et al., Micros. Res. Tech. 42:281-294 (1998); Chan, Science 5385:2016-2018 (1998); Cronenwett et al., Science 5376:540-544 (1998) and Landin et al., Science 5361:262-264 (1998)). Such Quantum Dots can be used as a detectable label or can be used as a control or tracer during an assay.

“Manipulation” refers to moving or processing of a moiety, which results in one-, two- or three-dimensional movement of the moiety, in a chip format, whether within a single chip or between or among multiple chips. Non-limiting examples of the manipulations include transportation, focusing, enrichment, concentration, aggregation, trapping, repulsion, levitation, separation, isolation or linear or other directed motion of the moieties, particularly in a magnetic field.

A “sample” is any fluid from which components are to be separated or analyzed. A sample can be from any source, such as an organism, group of organisms from the same or different species, from the environment, such as from a body of water or from the soil, or from a food source or an industrial source. A sample can be an unprocessed or a processed sample. A sample can be a gas, a liquid, or a semi-solid, and can be a solution or a suspension. A sample can be an extract, for example a liquid extract of a soil or food sample, an extract of a throat or genital swab, or an extract of a fecal sample. A sample can include, but is not limited to, a blood sample, white blood cells, red blood cells, neoplastic cells, malignant cells, stem cells, progenitor cells or an etiological agent. A sample can be any fluid sample, such as an environmental sample, including air samples, water samples, food samples, and biological samples, including extracts of biological samples. Biological samples can be blood, serum, saliva, urine, semen, ocular fluid, extracts of nasal swabs, throat swabs, or genital swabs or extracts of fecal material. Biological samples can also be samples of organs, tissues, or cell cultures, including both primary cultures and cell lines. A preferred sample is a blood sample.

A “blood sample” as used herein can refer to a processed or unprocessed blood sample, i.e., it can be a centrifuged, filtered, extracted, or otherwise treated blood sample, including a blood sample to which one or more reagents such as, but not limited to, anticoagulants or stabilizers have been added. A blood sample can be of any volume, and can be from any subject such as an animal or human. A preferred subject is a human. A blood sample can be any blood sample, recently taken from a subject, taken from storage, or removed from a source external to a subject, such as clothing, upholstery, tools, etc. A blood sample can therefore be an extract obtained, for example, by soaking an article containing blood in a buffer or solution. A blood sample can be a maternal blood sample. A blood sample can be unprocessed, processed, or partially processed, for example, a blood sample that has been centrifuged to remove serum, dialyzed, subjected to flow Cytometry, had reagents added to it, etc. A blood sample can be of any volume. For example, a blood sample can be less than five microliters, or more than 5 liters, depending on the application.

A “white blood cell” is a leukocyte, or a cell of the hematopoietic lineage that is not a reticulocyte or platelet and that can be found in the blood of an animal. Leukocytes can include lymphocytes, such as B lymphocytes or T lymphocytes. Leukocytes can also include phagocytic cells, such as monocytes, macrophages, and granulocytes, including basophils, eosinophils and neutrophils. Leukocytes can also comprise mast cells.

A “red blood cell” is an erythrocyte.

A “nucleated red blood cell” is a precursor to a red blood cell and are generally observed in newborn infants. The presence of nucleated red blood cells in adult peripheral blood generally indicates disease.

A “neonatal cell” is any cell produced by a newborn. A neonatal cell is generally produced within twenty eight days following birth.

A “fetal cell” is any cell produced by a fetus.

“Neoplastic cells” refers to abnormal cells that grow by cellular proliferation more rapidly than normal and can continue to grow after the stimuli that induced the new growth has been withdrawn. Neoplastic cells tend to show partial or complete lack of structural organization and functional coordination with the normal tissue, and may be benign or malignant.

A “malignant cell” is a cell having the property of locally invasive and destructive growth and metastasis.

A “stem cell” is an undifferentiated cell that can give rise, through one or more cell division cycles, to at least one differentiated cell type.

A “progenitor cell” is a committed but undifferentiated cell that can give rise, through one or more cell division cycles, to at least one differentiated cell type. Typically, a stem cell gives rise to a progenitor cell through one or more cell divisions in response to a particular stimulus or set of stimuli, and a progenitor gives rise to one or more differentiated cell types in response to a particular stimulus or set of stimuli.

An “etiological agent” refers to any etiological agent, such as a bacteria, virus, parasite or prion that can infect a subject. An etiological agent can cause symptoms or a disease state in the subject it infects. A human etiological agent is an etiological agent that can infect a human subject. Such human etiological agents may be specific for humans, such as a specific human etiological agent, or may infect a variety of species, such as a promiscuous human etiological agent.

“Subject” refers to any organism, such as an animal or a human. An animal can include any animal, such as a feral animal, a companion animal such as a dog or cat, an agricultural animal such as a pig or a cow, or a pleasure animal such as a horse.

“Separation” is a process in which one or more components of a sample is spatially separated from one or more other components of a sample. For example, a separation can be performed such that one or more moieties or moieties of interest are translocated to one or more areas of a separation apparatus such as a chip and optionally at least some of the remaining components are translocated away from the area or areas where the one or more moieties of interest are translocated to and/or retained in. Alternatively, a separation can be performed in which one or more moieties are retained in one or more areas and optionally at least some or the remaining components are removed from the area or areas. Alternatively, one or more components of a sample can be translocated to and/or retained in one or more areas and optionally one or more moieties can be removed from the area or areas and optionally collected. It is also possible to cause one or more moieties to be translocated to one or more areas and one or more moieties of interest or one or more components of a sample to optionally be translocated to one or more other areas. Separations can be achieved using physical, chemical, electrical, or magnetic forces. Examples of forces that can be used in separations are gravity, mass flow, dielectric forces, and electromagnetic forces.

“Capture” is a type of separation in which one or more moieties or moieties of interest is retained in one or more areas of a chip. A capture can be performed using a specific binding member that binds a moiety of interest with high affinity. The specific binding member can be reversibly or irreversibly bound to a solid support, or a portion of a solid support, such as a portion of a chip.

An “assay” is a test performed on a sample or a component of a sample. An assay can test for the presence of a component, the amount or concentration of a component, the composition of a component, the activity of a component and the like. Assays that can be performed in conjunction with the compositions and methods of the present invention include biochemical assays, binding assays, cellular assays, and genetic assays.

A “reaction” is a chemical or biochemical process that changes the chemical or biochemical composition of one or more molecules or compounds or that changes the interaction of one or more molecules with one or more other molecules or compounds. Reactions of the present invention can be catalyzed by enzymes, and can include degradation reactions, synthetic reactions, modifying reactions or binding reactions.

A “binding assay” is an assay that tests for the presence or concentration of an entity by detecting binding of the entity to a specific binding member, or that tests the ability of an entity to bind another entity, or tests the binding affinity of one entity for another entity. An entity can be an organic or inorganic molecule, a molecular complex that comprises, organic, inorganic, or a combination of organic and inorganic compounds, an organelle, a virus, or a cell. Binding assays can use detectable labels or signal generating systems that give rise to detectable signals in the presence of the bound entity. Standard binding assays include those that rely on nucleic acid hybridization to detect specific nucleic acid sequences, those that rely on antibody binding to entities, and those that rely on ligands binding to receptors.

A “biochemical assay” is an assay that tests for the presence, concentration, or activity of one or more components of a sample.

A “cellular assay” is an assay that tests for the presence of a cell such as a cell that has been separated or captured from a sample or is an assay that tests for a cellular process, such as, but not limited to, a metabolic activity, a catabolic activity, an ion channel activity, an intracellular signaling activity, a receptor-linked signaling activity, a transcriptional activity, a translational activity, or a secretory activity.

“Cell separation” is a method that isolates a cell utilizing the cell's physical or chemical properties from a medium containing at least one cell. The medium may be a fluid, such as, but not limited to a saline solution, tissue culture medium, blood, or maternal blood. The cell's physical properties are any cellular properties that one skilled in the art may exploit to separate a cell from a solution such as, but not limited to, a cell's isolectric point (PI), size, density, granularity, or dielectric constant. A cell's chemical properties result from any chemical or biochemical groups on the cell surface such as antigen and receptors.

“Cell capture” is a method that captures a cell from a medium utilizing a physical or chemical interaction with the cell. A cell may be captured from a homogeneous solution such as a culture medium having one cell type or from a heterogeneous mixture such as blood or culture medium having more than one cell type. Capturing a cell may be performed utilizing long range interaction or short range interaction such as, for example, covalent binding, ionic binding, or vanderwaals interactions. Capturing may be performed using a variety of techniques including, but not limited to a target cell interacting with an antibody, a cell, or a compound.

A “genetic assay” is an assay that tests for the presence or sequence of a genetic element, where a genetic element can be any segment of a DNA or RNA molecule, including, but not limited to, a gene, a repetitive element, a transposable element, a regulatory element, a telomere, a centromere, or DNA or RNA of unknown function. As nonlimiting examples, genetic assays can use nucleic acid hybridization techniques, can comprise nucleic acid sequencing reactions, or can use one or more polymerases, as, for example a genetic assay based on PCR. A genetic assay can use one or more detectable labels, such as, but not limited to, fluorochromes, radioisotopes, or signal generating systems.

“Binding partner” refers to any substances that bind to moieties or moieties of interest with desired affinity or specificity. Non-limiting examples of the binding partners include moieties such as nucleic acid molecules, proteins, antibodies, receptors cells, cellular organelles, viruses, microparticles or an aggregate or complex thereof, or an aggregate or complex of molecules.

“Coupled” means bound by any appropriate methods. For example, a moiety can be coupled to a microparticle by specific or nonspecific binding. As disclosed herein, the binding can be covalent or noncovalent, reversible or irreversible.

A “specific binding member” is one of two different molecules having an area on the surface or in a cavity which specifically binds to and is thereby defined as complementary with a particular spatial and polar organization of the other molecule. A specific binding member can be a member of an immunological pair such as antigen-antibody, can be biotin-avidin or biotin streptavidin, ligand-receptor, nucleic acid duplexes, IgG-protein A, DNA-DNA, DNA-RNA, RNA-RNA, and the like.

A “nucleic acid molecule” is a polynucleotide. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases, such as 2-aminoadenine, and the like. A nucleic acid molecule of the present invention can have linkages other than phosphodiester linkages. A nucleic acid molecule of the present invention can be a peptide nucleic acid molecule, in which nucleobases are linked to a peptide backbone. A nucleic acid molecule can be of any length, and can be single-stranded, double-stranded, or triple-stranded, or any combination thereof.

A “detectable label” is a compound or molecule that can be detected, or that can generate a readout, such as fluorescence, radioactivity, color, chemiluminescence or other readouts known in the art or later developed. The readouts can be based on fluorescence, such as by fluorescent labels, such as but not limited to, Cy-3, Cy-5, phycoerythrin, phycocyanin, allophycocyanin, FITC, rhodamine, or lanthanides; and by fluorescent proteins such as, but not limited to, green fluorescent protein (GFP). The readout can be based on enzymatic activity, such as, but not limited to, the activity of beta-galactosidase, beta-lactamase, horseradish peroxidase, alkaline phosphatase, or luciferase. The readout can be based on radioisotopes (such as ³³P, ³H, ¹⁴C, ³⁵S, ¹²⁵I, ³²P or ¹³¹I). A label optionally can be a base with modified mass, such as, for example, pyrimidines modified at the C5 position or purines modified at the N7 position. Mass modifying groups can be, for examples, halogen, ether or polyether, alkyl, ester or polyester, or of the general type XR, wherein X is a linking group and R is a mass-modifying group. One of skill in the art will recognize that there are numerous possibilities for mass-modifications useful in modifying nucleic acid molecules and oligonucleotides, including those described in Oligonucleotides and Analogues: A Practical Approach, Eckstein, ed. (1991) and in PCT/US94/00193.

A “signal producing system” may have one or more components, at least one component usually being a labeled binding member. The signal producing system includes all of the reagents required to produce or enhance a measurable signal including signal producing means capable of interacting with a label to produce a signal. The signal producing system provides a signal detectable by external means, often by measurement of a change in the wavelength of light absorption or emission. A signal producing system can include a chromophoric substrate and enzyme, where chromophoric substrates are enzymatically converted to dyes that absorb light in the ultraviolet or visible region, phosphors or fluorescers. However, a signal producing system can also provide a detectable signal that can be based on radioactivity or other detectable signals.

The signal producing system can include at least one catalyst, usually at least one enzyme, can include at least one substrate, may include two or more catalysts and a plurality of substrates, and may include a combination of enzymes, where the substrate of one enzyme is the product of the other enzyme. The operation of the signal producing system is to produce a product that provides a detectable signal at the predetermined site, related to the presence of label at the predetermined site.

In order to have a detectable signal, it may be desirable to provide means for amplifying the signal produced by the presence of the label at the predetermined site. Therefore, it will usually be preferable for the label to be a catalyst or luminescent compound or radioisotope, most preferably a catalyst. Preferably, catalysts are enzymes and coenzymes that can produce a multiplicity of signal generating molecules from a single label. An enzyme or coenzyme can be employed which provides the desired amplification by producing a product, which absorbs light, for example, a dye, or emits light upon irradiation, for example, a fluorescers. Alternatively, the catalytic reaction can lead to direct light emission, for example, chemiluminescence. A large number of enzymes and coenzymes for providing such products are indicated in U.S. Pat. Nos. 4,275,149 and 4,318,980, which disclosures are incorporated herein by reference. A wide variety of non-enzymatic catalysts that may be employed are found in U.S. Pat. No. 4,160,645, issued Jul. 10, 1979, the appropriate portions of which are incorporated herein by reference.

The product of the enzyme reaction will usually be a dye or fluorescers. A large number of illustrative fluorescers are indicated in U.S. Pat. No. 4,275,149, which disclosure is incorporated herein by reference.

Other technical terms used herein have their ordinary meaning in the art that they are used, as exemplified by a variety of technical dictionaries.

I. A Platform that Includes a Coating Film and Channel Structures

A first aspect of the present invention is a platform that includes: a surface, a coating film and a channel structure. Preferably, the coating film defines in part the channel structure and more preferably the platform comprises a microchip, such as a biochip. The platform is preferably a structure that forms one or more microchips, such as a wafer, but that is not a requirement of the present invention. The platform can be made of any appropriate material, such as, but not limited to silica, glass, quartz, fused silica, polymer, plastic, metal, metal oxide, PTFE, polysilicon, silicon nitride, ceramic, composit or carbon.

a. Surface

The platform can include at least in part a surface, which is coated at least in part by a coating film. The surface can be the same or different material as the platform, and the surface can be made of any appropriate material, such as but not limited to at least in part silica, glass, quartz, fused silica, polymer, plastic, metal, metal oxide, PTFE, polysilicon, silicon nitride, ceramic, composit or carbon. The surface can be a layer upon the platform structure, such as a layer of material deposited on the platform by way of, for example, sputtering or other appropriate methods of deposition. In one aspect of the present invention, the surface and the platform are the same.

The surface can include additional structures, such as elements that are useful in biological or chemical reactions, assays or manipulations, such as manipulations of particles such as cells. In one aspect of the present invention, the surface or platform can include a magnetic element, an electromagnetic element, an acoustic element, a dielectric element or combinations thereof. These additional elements can be provided on, within or external to the surface or the platform. Preferably, the additional elements are provided within the surface. The additional elements can be arranged or provided in any appropriate location below, within or on the surface, but are preferably arranged to facilitate biological or chemical reactions, assays or manipulations. The additional structures can be provided in one or more planes within the surface or platform. For example, electromagnetic elements can be provided in one plane, acoustic elements in a second plane and dielectric elements in a third plane. These elements can be manufactured by a variety of methods known in the art, such as by masking and deposition of appropriate materials using methods in the microchip and electronic chip fields.

The platform can be of any appropriate size and shape, but is preferably a wafer or square having a length at its greatest width of between about 0.1 cm and about 100 cm, preferably between about 1 cm and about 10 cm. The platform can be of any appropriate thickness, but is preferably between about 0.1 mm and about 100 mm in thickness, preferably between about 1 mm and about 50 mm in thickness.

The surface can be co-extensive with the platform, but that is usually not the case. The surface can be of any appropriate size or shape, preferably between about 10 micrometers and about 10 centimeters in length at its greatest width, and more preferably between about 1 mm and about 1 cm. The surface can be of any appropriate thickness, preferably between about 0.1 micrometers and about 10 centimeters in thickness, and more preferably between about 10 micrometers and about 1 cm in thickness or between about 1 mm and about 100 mm in thickness.

b. Coating Film

The coating film can be of any appropriate material, but is preferably polymeric. The coating film can include at least in part polymers such as a homopolymer, copolymer, cross-linked polymer, partially polymerized polymer or a cross-linked polymer network. The coating film can include at least in part either or both of a hydrophobic polymer or a hydrophilic polymer. The relative hydrophobicity or hydrophilicity can be determined by established methods. For example, hydrophilic surfaces are more water wetable than are hydrophobic surfaces. Also, the contact angle of a drop of water on a surface is proportional to the hydrophobicity or hydrophilicity of the surface. In the latter case, the smaller the contact angle, the more hydrophiclic the surface. The hydropobicity or hydrophilicity of the coating film is a matter of choice based on the intended use of the assay. For example, hydrophilic polymers are generally more appropriate for biological reactions whereas hydrophobic polymers are generally more appropriate for chemical reactions. However, hydrophobic or hydrophilic polymers can be appropriate for both biological reactions and chemical reactions. Preferred polymers include, but are not limited to polyethyleneglycol, polyurethanes, polyacrylates, polyacrylamides, polymethylacrylamide, polyvinyl alcohol, polyvinylpyrolidone, polyamino acids, polysaccharides and polysiloxanes, polybutadine and epoxy resins.

In one preferred aspect of the present invention, the coating film is biocompatible, such as a biocompatible polymer. Biocompatable polymers are known in the art and do not substantially interfere with biological processes, binding reactions or substantially alter biological moieties. In one preferred aspect of the present invention, biocompatible polymers do not substantially adhere with or absorb with biological moieties. This property is particularly appropriate for biological binding reactions where it is undesirable for biological moieties to adhere to surfaces such that the signal to noise ratio of an assay is altered or the sensitivity of the assay is adversely impacted because analytes or reagents become immobilized on a structure and thus do not partake in a reaction or are not detected. For example, certain polymers, such as polystyrene used in immunoassays, is preferably “blocked” or absorbed with a blocking protein, such as serum albumin, to prevent non-specific absorption of reagents during an assay. In one aspect of the present invention, the need for such blocking is diminished due to the propensity of certain biologically compatible polymers not to absorb or non-specifically immobilize biological moieties. Polyethylene glycol (PEG) is a particularly preferred biocompatible polymer. Other biocompatible polymers include PMMA. However, this characteristic of biologically compatible polymers is not a requirement of the present invention. In assays that utilize platforms of the present invention, it may be preferable to contact the platform, particularly the channel structures or other portions of the platform that come in contact with biological moieties to be pre-treated with a blocking agent, such as serum albumin, such as bovine serum albumin, as such methods are established in the art.

The coating film can be of any appropriate size and dimensions of length or width, but is preferably between about 10 micrometers and about 10 centimeters in length or width, more preferably between about 1 mm and about 1 cm in length or width.

The coating film can be of any appropriate thickness, but is preferably between about 0.1 micrometers and about 10 millimeters in thickness, more preferably between about 1 micrometer and about 1 millimeter in thickness or between about 10 micrometers and about 100 micrometers in thickness.

1. Biological Group

In one aspect of the present invention, particularly where the platform of the present invention is intended to take part in a biological assay, the coating film includes at least in part a biological group. The biological group can be localized anywhere on the coating film, but is preferably located on at least one structure that defines a channel structure. Any biological group can be provided on or within the coating film, such as a polypeptide, antibody, receptor, protein, nucleic acid, small molecule, carbohydrate, lipid or combinations thereof. Biological groups can be provided on or within the coating film during manufacture, such as by entrapment of a biological group within a polymer matrix, or be coated on a coating film such as by absorption, cross-linking biological groups to each other, cross-linking biological groups to the coating film, or chemical linking to the coating film. In one aspect of the present invention, biological groups are provided substantially throughout at least a portion of the coating film.

In one preferred aspect of the present invention, the biological groups can interact with a biological moiety or chemical moiety. Such interactions can be by short-range interactions such as by electrostatic interactions, ionic interactions, hydrogen bonding or hydrophobic interactions.

These interactions can be non-specific in nature, such as in the instance where the platform of the present invention is used to separate moieties based on their general physical and chemical properties, such as, by analogy, ion exchange chromatography or high performance liquid chromatography where a stationary phase having a physical or chemical characteristic can modulate the mobility of a moiety along a length of stationary phase.

The interactions between a biological group and a biological moiety or chemical moiety can also be specific in nature, such as through specific binding interactions as they are known in the art. A wide variety of specific binding interactions are known, such as nucleic acid—nucleic acid interactions, nucleic acid—protein interactions, antigen—antibody interactions, receptor ligand interactions or protein—small molecule interactions to name representative interactions. Many of these interactions have been used in the field of affinity chromatography for separations of moieties in a sample.

2. Chemical Group

The coating film can also include at least in part a chemical group. Coating films can also include chemical groups and biological groups, though they need not be provided at the same locations on the coating film. Chemical groups when present can have a variety of functions, such as groups that can be used to immobilize other chemical moieties or biological moieties, such as through cross-linking such as through the formation of covalent bonds between the chemical group and biological moieties or chemical moieties.

Chemical groups can interact with a chemical moiety or biological moiety by short-range interactions or by covalent bonding. For example, short-range interactions include but are not limited to electrostatic interactions, ionic interactions, hydrogen bonding or hydrophobic interactions. Covalent linking can take place by way of appropriate chemical reactions. Preferred chemical groups include, but are not limited an alkyl group, a charged group, a positively charged group, a negatively charged group, small molecules or combinations thereof.

The chemical groups can be provided within or on the coating film. Preferably, the chemical groups are provided on at least one surface that defines a channel structure. The chemical groups can be entrapped within the coating film or be chemically linked to the coating film by way of short-range interactions or chemical bonds. In one aspect of the present invention, the chemical groups are provided substantially throughout the coating film. Chemical groups can also be provided on the surface of the coating film such as by coating methods as they are known in the art. For example, channel structures within a coating film can be contacted with a chemical group such that the chemical group becomes localized at such locations. Chemical groups can become immobilized using short-range interactions or covalent bonding.

C. Particles

In one preferred aspect of the present invention, the coating film can also include particles. The particles can perform a variety of functions, such as supporting the coating film that can allow the coating film to have greater stability and greater thickness than when such particles are not present. This is particularly true in the instance where hydrophilic polymers, particularly hydrogels, are used in the coating film.

When hydrogels, particularly biocompatible hydrogels such as PEG and polyhdroxethyl acrylate (PHEA), are used as the coating film, swelling may occur to an extent such that channel structures are narrowed or closed. Particles provided in the coating film can reduce this phenomenon by providing structural support so that the shape of the coating film and channel structures are stabilized.

The particles can be provided in any appropriate concentration within the coating film and need not be homogeneously distributed throughout the coating film. In volume: volume percentages, the concentration of particles within the coating film is preferably between about 0.1% and about 99.9%, more preferably between about 1% and about 75% or between about 5% and about 50% and most preferably between about 10% and about 30%. The volume or number of particles in a coating film is a matter of choice based on the desired characteristics of the coating film. For example, by increasing the volume or number of particles within the coating film, the coating film will tend to be more ridged and more stable. Also, if the particles include biological groups or chemical groups, increasing the volume or number of particles increases the surface area of particles present in the coating film, which in turn increases the load of biological groups or chemical groups in the coating film. Furthermore, increasing the number or volume of particles in the coating film tends to increase the surface area of particles that are presented on a surface of a coating film, such as surfaces of coating film that are presented on a channel structure.

The particles can be of any appropriate material, shape or size. The particles can be homogeneous or heterogeneous in shape, size or composition. Particles are preferably generally spherical in nature, but that is not a requirement of the present invention. For example, particles may have sharp edges or rounded edges, or a combination thereof. Particles preferably include at least in part at least one of the following materials: glass, silica, quartz, fused glass, polymer, Sephadex™, metal oxide, polystyrene, PMMA, plastic, dextran, agarose, polyamide, polysaccharides or polyimide. The particle can be magnetic or be a detectable label, such as a latex bead, a colored particle or a light emitting particle, such as a Quantum Dot or a particle containing one or a population of Quantum Dots.

The size of the particles is one of choice based on the characteristics of the coating film and the intended use of the platform of the present invention. Preferably, the particles are on average between about 0.05 micrometers or about 0.1 micrometer in diameter to about 50 micrometers or about 500 micrometers in diameter, more preferably between about 1 micrometer and about 10 micrometer in diameter.

In one aspect of the present invention, the particles are preferably biocompatible. This is particularly true where the platform of the present invention is to be used for biological assays. Further more, the particles are at least partially transparent to electromagnetic radiation used for photocuring, particularly light in the UV or visible spectrums. Particles having this characteristic are particularly preferred because these types of particles would tend to scatter light less and thus assist in localizing polymerization to particular loci. Preferred particles are made of UV-transparent or UV-semi-transparent materials, such as quartz, fused silica, polypropylene, polyethylene, selected metal oxides and selected ceramic materials.

1. Biological Group

In one aspect of the present invention, at least a portion of a population of particles can include a biological group. The biological group is preferably immobilized on the surface of the particle, but can be integrated throughout the particle as well. In a preferred aspect of the present invention, a biological group can be immobilized on the surface of a particle, such as through absorption, passive absorption, cross-linking of biological groups to each other or to a particle, or by linking a biological group to a particle, such as by covalent linking. In the latter case, it is preferable for a particle to have functional groups thereon that can facilitate the formation of a covalent bond between the particle and the biological group. Not all particles in a population of particles need have such biological groups. Particles that include biological groups can be distributed equally or unequally through the coating film. In the alternative, such particles can be placed on the surface of a coating film, such as by using deposition methods. It is preferred that particles that include biological groups be concentrated on exposed surfaces of the coating film, such as channel structures, but that is not a requirement of the present invention.

2. Chemical Group

In one aspect of the present invention, at least a portion of a population of particles can include a chemical group. The chemical group is preferably immobilized on the surface of the particle, but can be integrated throughout the particle as well. In a preferred aspect of the present invention, a chemical group is linked to the particle, preferably by covalent linkages. It is preferred that the chemical groups be chemically reactive, such as by having functional groups that are capable of a variety of chemical reactions, such as forming linkages such as covalent linkages to chemical moieties, biological moieties, polymers or monomers. Not all particles in a population of particles need have such chemical groups. Particles that include chemical groups can be distributed equally or unequally through the coating film. In the alternative, such particles can be placed on the surface of a coating film, such as by using deposition methods. It is preferred that particles that include chemical groups be concentrated on exposed surfaces of the coating film, such as channel structures, but that is not a requirement of the present invention.

In one aspect of the present invention, the chemical groups on a particle can be used to bond with a biological group or biological moiety. In that way, a particle that includes a biological group is formed.

d. Channel Structures

The channel structures of the platforms of the present invention are formed at least in part by the coating film. The channel structures can be open channels or closed channels. The open channels have at lest one side open, such as, by analogy, an irrigation channel used in agriculture. Closed channels do not have at least one side open, such as, by analogy, a pipe that is used to carry water. Open channels can be made into closed channels by the use of a covering structure, such as a cover slip or similar structure. In the alternative, a covering structure can be deposited on the platform using microfabrication methods or polymerization methods, particularly those used in the contact lens manufacturing arts. The covering structure can include polymers, such as the materials used to make the coating layer.

The channel structures are preferably defined at least in part by at least one of the following: the coating film, the surface, the platform or a covering structure. Preferably, open channels are defined by the coating film, but that is not a requirement of the present invention. In that way, the channel structures are formed by the coating film and include any biological groups or chemical groups therein or thereon. In the alternative, the surface and/or platform can define at least a portion of a channel structure. For a closed channel structure, at least a portion of a channel is preferably defined by a covering structure. It is preferred that at least a portion of such closed channels are defied by the coating film, particularly when biological groups or chemical groups in or on the coating film are presented to the channel structure.

In one preferred aspect of the present invention, at least a portion of a channel structure is defined by selective polymerization of said coating film. As discussed herein, one method of manufacture of the platforms, microchips and biochips of the present invention is to provide a polymerizable composition upon a surface or platform in a thin layer. The thin layer of polymerizable material can be made using established methods, such as spin casting or thin layer deposition, such as through microfluidic application of the polymerizable composition. The polymerizable composition preferably includes a polymerization initiator, such as a photoinitiator. A mask, such as a photomask, can be place between a light source and the polymerizable composition such that the polymerizable composition is selectively polymerized at locations on the surface/platform. Unpolymerized material is removed, such as through washing. The removed unpolymerized material forms channel structures.

Channel structures can form a variety of patterns within a coating film. For example, the channels can form serpentine patterns, linear patterns, islands, coiled patterns, curved patterns, saw-toothed patterns, switchback patterns or combinations thereof. In one aspect of the present invention, particularly where moieties are separated along the length of a channel, it is preferred that the channel structure be compact on the surface. In this instance, purely linear channels are not particularly desirable. Thus, other patterns can be used to increase the length of the channel structure without increasing the size of a chip. Preferred patterns include coils and switchbacks, but other patterns can also be used.

The shape of a channel structure in cross-section can be the same or different throughout the length of the channel structure. Preferred cross sections include, but are not limited to substantially square, substantially oval, substantially crescent, substantially half-circle or substantially rectangular. The shape of the channel in cross-section can be determined using a variety of methods. For example, the width of the channel structure can be determined by the use of a mask. The depth of the channel by the duration of exposure to polymerizing events such as exposure to light or the concentration or type of polymerizing initiator used.

Furthermore, the channel structures, including wells or chambers, can optionally form islands. These channel structures, wells, chambers and islands can form surface patterns analogous to those on microarray devices.

e. Additional Structures

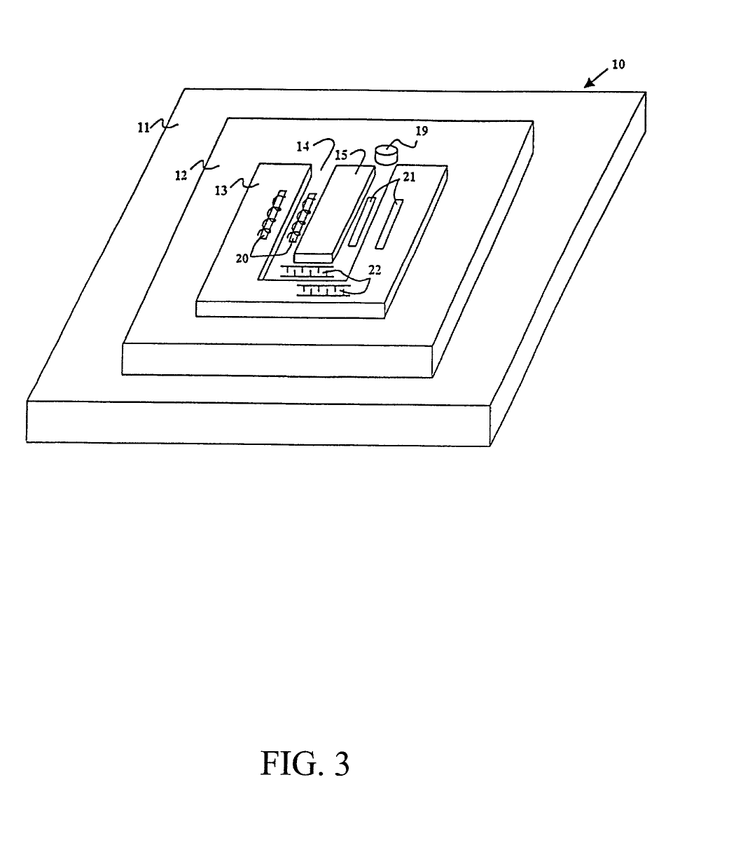

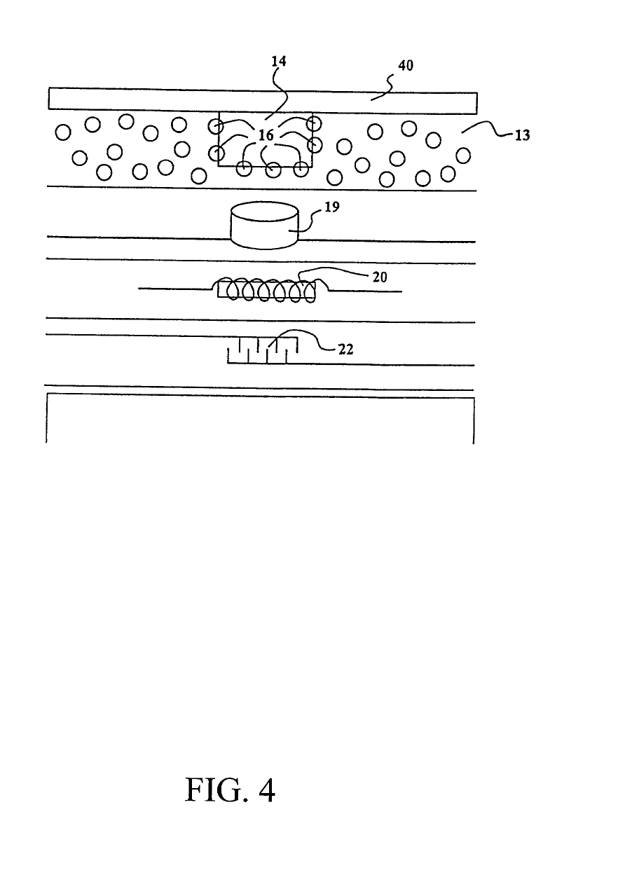

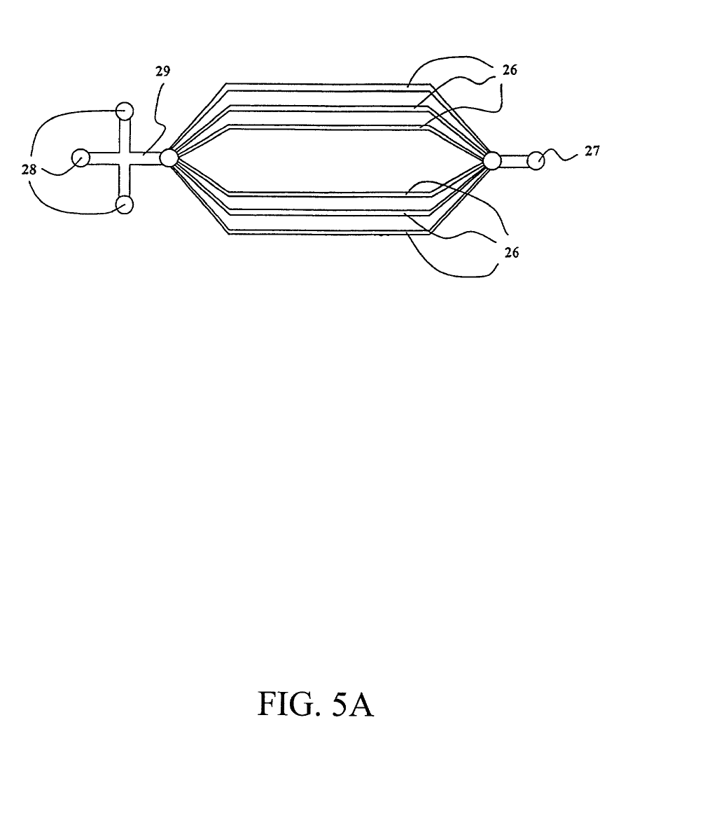

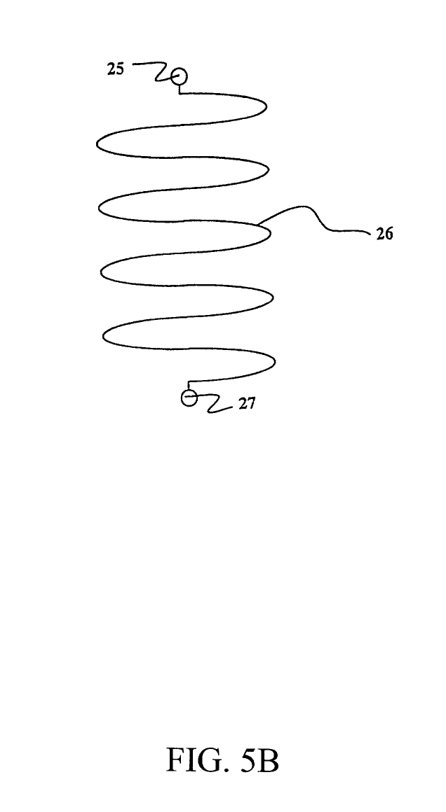

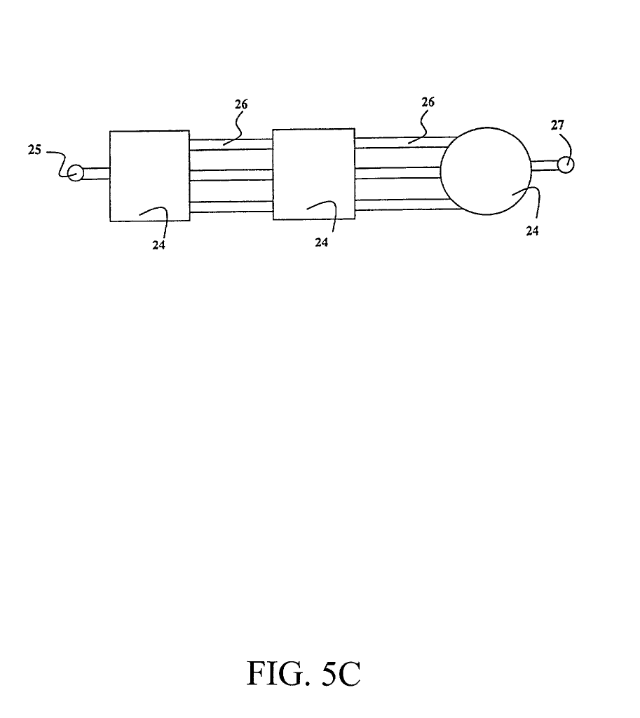

The platform of the present invention can also include a variety of additional structures, such as a magnetic element, an electromagnetic element, an acoustic element or a dielectric element. Each of these structures can be provided on a platform at a desired location, such as on or within a platform, a surface or a coating film. These structures can be completely buried or partially exposed to the surface of the platform, surface or coating film. These structures are particularly useful in biochip or laboratory on a chip applications, where particles, including cells, are moved. As shown in FIG. 3 and FIG. 4, the additional elements are preferably provided below the coating film and oriented to be aligned with a channel structure.

II. A Method of Making a Platform and Platforms Made by Such Methods

A second aspect of the present invention is a method of making a platform that includes at least one channel structure. The method includes: providing a surface and contacting the surface with a polymerizable composition. The polymerizable composition preferably includes: unpolymerized polymer subunits and at least one polymerization initiator. The polymerizable composition is preferably selectively polymerized at loci to form a platform that includes a polymerized layer that defines at least in part at least one channel structure.

a. Surface

A surface is provided upon which a coating film will be made. The surface can be of any appropriate material, but is preferably at least in part silica, glass, quartz, fused quartz, polymer, plastic, metal, metal oxide, PTFE, polysilicon, silicon nitride, ceramic, composit or carbon. Preferred materials are those that are routinely used in electronic chip manufacture, particularly those materials that are biocompatible and optionally those that do not substantially fluoresce. The surface can include a variety of additional structures, such as, for example, a magnetic element, an electromagnetic element, an acoustic element, a dielectric element or combinations thereof. These elements can be provided in a single or multiple layers and are preferably provided in orientations and configurations that will ultimately align with channel structures. The orientation and alignment of these additional structures preferably corresponds to a chemical reaction, biological assay or other assay that will utilize the forces generated by the additional structures during the course of a procedure.

b. Polymerizable Composition

A polymerizable composition is then provided on the surface. The polymerizable composition is preferably a viscous liquid, but can also be a suspension or emulsion. The polymerizable composition includes unpolymerized polymer subunits comprise monomers, macromonomers or combinations thereof. In one aspect of the present invention, the unpolymerized polymer subunits comprise partially polymerized polymer. Upon polymerization, the unpolymerized polymer subunits polymerize to form a homopolymer, copolymer, cross-linked polymer or a cross-linked polymer network. The resulting polymer can be a hydrophobic polymer or a hydrophilic polymer. Particularly for applications were biological groups or biological moieties are present in the polymerizable composition, the unpolymerized polymer subunits are preferably biocompatible. A wide variety of polymerizable units and compositions are appropriate for use in the present invention. Preferred unpolymerized polymer subunits include subunits of at least one polymer selected from the group consisting of acrylic, methacrylic, vinylbenzyl, vinyl, epoxy, polymers comprising pendant alpha, beta unsaturated ketones, polymers comprising pendant chalcone moieties and polymers comprising cinnamates.

The type, concentration and amount of various polymerizable materials can be determined based on the desired characteristics of the polymerized product. By increasing the concentration of polymerizable materials, the resulting polymer tends to be stiffer and stronger. A still stronger resulting polymer can be achieved by including polymerizable materials that form cross-linked polymers or polymer networks. One preferred type of polymerizable composition includes free-radical polymerizable monomers with a photocrosslinkable function group. These types of polymerizable compositions have been described in the art (Subramanian et al., European Polymer Journal 36:2343-2350 (2000)) and include a polymerizable methacryloyl group and pendant chalcone units.

C. Polymerization Initiator

The polymerizable composition preferably includes a defined polymerization initiator. The polymerization initiator is preferably one that is activated by a defined activity, energy or force, such that thermal events or light. Thus, photointiators or thermal initiators are preferred. Photoinitiators are particularly preferred because light can be particularly focused and modulated, particularly in the instance of laser light. Preferred photoinitiators include 2,2-dimethoxy-2-phenyl acetophenone, benzophenone, mono-acylphosphineoxides (MAPO), Bisacylphosphineoxides (BAPO) and anthraquinone. Commercially available photoinitiators such as Irgacure® 1300 or 2959 from Ciba (Tarrytown, N.Y.) can also be used. Initiators can be provided in the polymerizable composition at concentrations that produce a polymer of desired characteristics. For example, a high concentration of initiator tends to induce a stiffer and stronger polymer, but also tends to lead to a bleed-over effect where polymerization can occur in masked areas. Preferred concentrations of polymerization initiators is between about 0.1% and about 10% weight/volume.

d. Selectively Polymerizing

One preferred aspect of the present invention is the selective polymerization of the polymerizable composition to form structures, such as channel structures. Selective polymerization preferably includes a localized initiation of said at least one polymerization initiator. In the case of photoinitiators of polymerization, localization of electromagnetic radiation, UV light or laser light can be accomplished with the appropriate optics. For commonly used photoinitiators, the electromagnetic radiation used is preferably between about 180 nanometers and about 600 nanometers. The particular wavelength used can be chosen based on the characteristics of the photoinitiator. Localization of electromagnetic radiation can also be accomplished using masking. Several types of masking are available. Preferred maskings are photomasking, transparency masking, transparency areas or windows. Masking materials and methods particularly useful in the present invention are those used in the manufacture of semiconductor chips.

A variety of photocuring methods are known in the art that are applicable to the present invention. For example, methods for UV curing through semi-transparent materials are available (Skinner et al., RadTech International North America 98 Conference, Chicago, Ill. Apr. 19-22, 1998, referring to DVD disk technologies). These methods utilize high intensity light and selected spectral outputs. Also, pigmented systems pose challenges for polymerization applications. For example, in UV-powder coating technologies preferably utilize bis-acyl phosphine oxide (BAPO) photoinitiators at depth by free radical polymerization. This has been attributable to absorption by BAPO of blue light that is generally better able to penetrate further through a polymerizable layer than is UV light. Preferred photoinitiators include those of the alpha-hydroxy ketone (AHK class, such as bis(2,4,6-trimethylbenzoyl)-phenylphosphine oxide and 1-[4-(2-hydroxyethoxy)-pheyl]-2-hydroxy-2-methyl-1-propane-1-one (also known as lrgacure 819 and lrgacure 2959, respectively). The use of films or colored materials can facilitate selective polymerization in the present invention. For example, films that have portions that allow and do not allow transmission of electromagnetic radiation can be used for masking. In addition, the use of colors in such films or in the polymerizable compositions, such as in the form of particles, inks or dyes, can be used to selectively or preferentially localize a polymerization event to a locus, such as an identified locus.

In the present invention, where the coating film can be relatively thick, the selection of appropriate polymers and initiators is important. For example, deep UV penetration may be required for these types of applications. In the alternative, as discussed, visible-light absorbing initiators can be used. Preferably, the combination of polymer and initiator allow for electromagnetic radiation, such as UV or visible light, to penetrate deeply into or completely through the coating film. Preferably, the polymerizable solution is substantially transparent or transparent to the electromagnetic radiation used for the initiating event. This is also true of the particles. It is important for the particles, when present, not to substantially absorb or substantially scatter electromagnetic radiation used for initiation. Preferred materials for particles include quartz, glass, fused silica, polysaccharides or other materials that are UV-transparent or semi-UV-transparent.

1. Biological Group

In one aspect of the present invention, the polymerizable composition can optionally include one or more biological groups. In this aspect of the present invention, the polymerizable composition and the resulting polymeric coating film, and preferably intermediates generated during polymerization methods, are biocompatible. In one aspect of the present invention, biological groups are provided homogeneously through the polymerizable composition that, when polymerized, results in the biological group being present substantially throughout said polymer coating. Alternatively, a composition that includes a biological group can be placed on the surface of the polymerizable composition such that the biological group is not distributed throughout the coating film but is rather provided at or near the surface of said coating film. In one aspect of the present invention, once a channel structure is formed, an additional thin layer of polymerizable composition that includes a biological group can be deposited on the coating film at desired locations, such as the entire surface or localized at channel structures. This composition can then be polymerized to result in a coating that includes a biological group.

2. Chemical Group

In one aspect of the present invention, the polymerizable composition can optionally include one or more chemical groups. In one aspect of the present invention, chemical groups are provided homogeneously through the polymerizable composition that, when polymerized, results in the chemical group being present substantially throughout said polymer coating. Alternatively, a composition that includes a chemical group can be placed on the surface of the polymerizable composition such that the chemical group is not distributed throughout the coating film but is rather provided at or near the surface of said coating film. In one aspect of the present invention, once a channel structure is formed, an additional thin layer of polymerizable composition that includes a chemical group can be deposited on the coating film at desired locations, such as the entire surface or localized at channel structures. This composition can then be polymerized to result in a coating that includes a chemical group.

3. Polymerizing Functional Group

In one aspect of the present invention, the polymerizable composition can optionally include at least one polymerizing functional group. The polymerizing functional group preferably can form a bond with a polymer, a monomer or a particle. A variety of polymerizing functional groups are known in the art and are commercially available. Preferred polymerizing functional groups include, but are not limited to, acrylics, methacrylics, vinylbenzyls, vinyls, epoxies, alpha/beta usaturated ketones, cinnamates, chalcone groups. The type and concentration of polymerizing functional groups used in a polymerizable composition can be determined based on the desired characteristics of the polymerized product. For example, the use of polymerizing functional groups and the use of such groups at higher concentrations tends to result in a polymer of greater stiffness and strength, particularly due to cross-linking events when they do occur. The greater strength and cross-linking of the polymer tends to result in a polymer that can trap a variety of materials, including particles, biological groups and chemical groups. The added strength also tends to allow the coating film that includes the polymerized product to be of greater thickness and durability.

e. Polymerized Layer

The polymerizable composition is then polymerized, preferably using selective polymerization. Any appropriate polymer can be used in the polymerized layer. Preferred polymers include, but are not limited to polyethyleneglycol, polyurethanes, polyacrylates, polyacrylamides, polymethylacrylamide, polyvinyl alcohol, polyvinyl prolidone, polyamino acids, polysaccharides, polysiloxanes or combinations thereof.

The resulting polymerized layer can include a variety of materials. If the polymerizable composition included biological groups, chemical groups or particles, then these materials are present in the polymerized layer. When present, channel structures formed by the selective polymerization preferably include biological groups, chemical groups or particles or portions thereof. In one aspect of the present invention, the chemical groups and/or biological groups are distributed throughout the polymerized layer, but that is not a requirement of the present invention.

In one aspect of the present invention, chemical groups or biological groups can be provided on the polymerized layer after polymerization. For example, chemical groups can be added to a polymerized layer by way of chemical reactions. In addition, biological groups can be absorbed onto a polymerized layer. In the alternative, a polymerized layer that includes chemical groups can be used to immobilize biological groups, such as through chemical reactions, such as the formation of covalent bonds with the biological group and the chemical group. These modifications can take place on the entire polymerized layer or at certain loci, such as loci that include channel structures. Preferably, chemical groups or biological groups are presented in the channel structure.

f. Particles

When present in the polymerizable composition, the resulting polymerized layer can include these particles. The particles in the polymerized layer tend to retain approximately the relative concentration (volume: volume) as in the polymerizable composition. However, some polymers can shrink or swell depending on the environment that they are in, thus this ratio can change. This is particularly true with hydrophilic polymers. The particles also tend to retain their physical characteristics, such as size and shape. The particles also tend to retain biological groups or chemical groups that were provided thereon in the polymerizable composition. However, polymerization is a process that can generate a variety of short-lived yet reactive compounds, such as free radicals. The compounds can modify the amount and character of biological groups or chemical groups provided on the particles in the polymerizable composition. Furthermore, the polymerizing functional groups, when provided in the polymerizable composition, can form a variety of structures. For example, polymerizing functional groups in the polymerizing composition can form cross-links between polymers or monomers and can form bonds with particles, particularly particles that include polymerizing functional groups. Particles that include polymerizing functional groups can form bonds with other particles or with polymers or monomers.

g. Channel Structures

Selective polymerization can result in the formation of channel structures. The channel structures are enhanced by removing unpolymerized materials or partially polymerized materials, such as through washing with an appropriate solution, such as an aqueous solution, such as a buffer. Open channels can be made into closed channels using a covering structure. The term “channel structure” includes wells and chambers as discussed herein.

Depending on the degree of polymerization and whether polymerization occurred at the interface of the surface and the polymerizable composition, the channel structures can in part be defined by the surface. However, if such polymerization did occur at that interface, then the open channel thus formed is defined by the polymerized layer, which forms the coating film. When is closed channel is formed, then the channel is preferably defined at least in part by the covering structure.

Depending on the conditions of polymerization, the degree of polymerization and the concentration of polymer subunits and polymerizing initiators, the resulting channel structures can have a variety of shapes in the cross section. Relatively incomplete polymerization characterized by low concentrations of polymer subunits, low amounts of cross linking, low concentrations of initiator and relatively short polymerization times results in channel shapes in the cross section that have more sharply defined walls tending more towards the perpendicular. More complete polymerization tends to result in channel structures in the cross section having shapes that are more oval, crescent or half-circle in shape. This difference in shape can be attributed to a bleed-through effect of initiator being active beyond its particular location. In addition, light scattering, particularly when particles are present in the polymerizable composition, can tend to lead to more complete polymerization.

The channel structures can be of any desires shape along their length and can be of different shapes and sizes along the length. Preferably, the channel structures are not linear in nature because many uses of the devices of the present invention utilize relatively long channel structures to, for example, separate materials, such as biological materials, in a sample. Thus, channels can take forms that increase the length of the channel without increasing the size of the surface. Preferred channel structure shapes that accomplish this goal include but are not limited to circular, coiled, curved, saw-toothed or switchback along at least a portion of a channel structure length. Furthermore, a channel structure can form an island structure. This type of structure is preferably formed when in the inlet of a channel structure and an outlet of a channel structure define an island.

h. Platform Made by a Method of the Present Invention

The present invention also includes a platform made by a method of the present invention. The platform can include or define a microchip or a biochip.

III. A Method of Separating Moieties Using a Platform

A third aspect of the present invention is a method of separating moieties that includes: providing a platform of the present invention, providing a sample containing moieties, contacting the platform with the sample, moving the sample through channels on the platform such that moieties within the sample are separated and optionally detecting at least one moiety. In one aspect of the present invention, the method: providing a platform of the present invention, providing a sample containing moieties, contacting the platform with a sample, moving the sample through channels on the platform such that moieties within the sample are separated and optionally manipulated and optionally detecting at least one moiety. The separation methods can include high performance liquid chromatography (HPLC), capillary electrophoresis (CE), and capillary electrochromatography (CEC) using the channels on the platform. The manipulation of sample moieties is preferably performed by applying appropriate external forces through means such as microfluidic devices or by applying appropriate electric or magnetic forces.

In this aspect, any appropriately configured microchip of the present invention can be utilized. Preferably, the biochip includes particles in the coating film, but that is not a requirement. Furthermore, the biochip preferably includes chemical groups or biological groups, preferably exposed to the channel structures that can include wells or chambers. The channel structure is preferably relatively long in length such that chemical moieties or biological moieties present in a sample can interact with the chemical groups or biological groups exposed on the surface of the channel structures. Depending on the affinity of the chemical moieties or biological moieties towards the chemical groups or biological groups on the channel structures, the chemical moieties or biological moieties progress along the length of the channel structure will be impeded due to interactions between such groups and moieties. For example, short range or covalent reactions can occur between the groups and moieties that can immobilize or impede with the progress of the moieties along the length of a channel structure or a portion thereof.

The channel structures can have any appropriate biological groups or chemical groups provided thereon. The selection of such groups provides a microchip that has physical, biological and chemical composition and structure that can be tailored to a particular separation method or system. For example, positively charged chemical groups can be used to impede the progress of negatively charged moieties along the path of a channel structure. Also, hydrophobic groups can be used to impede the progress hydrophobic moieties or moieties with at least a portion of hydrophobic characteristics. More selective structures can include channel structures that include specific binding members such as antibodies, receptors or active fragments thereof. These types of channel structures can impede or prevent the movement of moieties that interact with the specific binding members, particularly where the affinity or avidity of the moiety to the specific binding member is high. For example, metal chelating reagents such as Ni-NTA may be used to bind proteins fused with about 6 histidine residues. Similarly, glutathione bonded surface may be used to bind glutathione s-transferase (GST) fusion protein. In the alternative, ligands can be provided on the surface of channel structures such that specific binding members in a sample would tend to interact with the surface and thus impede the progress of movement of the specific binding member's progress along the path of a channel structure.

Furthermore, particles that are exposed to the surface of channel structures can have pores within them, such as with Sephadex™ so that the channel structures can act as molecule sieves such that the progress of smaller molecules along the path of a channel structure is impeded more than larger molecules. This phenomenon is believed to occur because smaller molecules can enter into the porous structure of the particles and thus have a longer path to take along a channel structure relative to larger molecules that do not or cannot enter the porous structures.

Combinations of porous structures with biological groups or chemical groups can add additional dimensions to the separation potential of the channel structures. For example, channel structures with positively charged groups and small pore size particles would tend to impede the progress of small negatively charged moieties in a sample relative to large positively charged moieties.

Moieties in a sample that are immobilized on a channel structure via short range interactions can be eluted using appropriate methods. For example, surfactants, salts, chaotropic agents or antichaotropic agents can be used to alter the chemical nature and the structure of water in an eluting buffer. The use of relatively high salt tends to disrupt ionic interactions while promoting hydrophobic interactions whereas the use of surfactants such as detergents tends to disrupt hydrophobic interactions. Salts and detergents can be combined to disrupt both hydrophobic interactions and ionic interactions. Furthermore, chaotropic agents tend to decrease the structure of water and disrupt hydrophobic interactions whereas antichaotropic agents tend to have the opposite effect. In addition, high salt concentration tends to promote hybridization of nucleic acid molecules to one another such as is known in the art of stringency for nucleic acid hybridization reactions.

The progress of moieties along the path of a channel structure can be monitored along the path of the channel structure or detected in the effluent from the channel structure. For example, detectors can be placed along the path of a channel structure to interrogate the contents of the channel during the course of a separation procedure. In one aspect of the present invention, spectrophotometic devices can be used to determine light scattering, light absorption, light emission or other light based detection phenomenon to determine the location and type of moiety at a location along a channel structure. Different moieties have different spectral signatures, such as absorption of light of various wavelengths. Fluorescent moieties exhibit excitation spectra and emission spectra that can be interrogated and detected. Particulates exhibit light scattering profiles as well. In addition, chromogenic moieties have spectral signatures that can be detected. Also, moieties can be labeled with detectable labels, such as chromogens, particulates, fluorophores or radioisotopes or radioprobes to facilitate detecting these moieties in the channel structure. In the alternative to detection of such moieties can be made at a point along the channel structure or at the end of the channel structure, such as by analogy a detector for the effluent from a column chromatography apparatus.

In operation, a sample is injected into channel structures on a microchip. The chemical and physical properties of the channel structures can impede the progress of certain moieties in the sample along the path of the channel structure based on the moieties chemical and physical characteristics. As the sample progresses along the channel structure, different populations of moieties tend to separate or band together. At or near the end of the channel structure, a detection device, such as a spectrophotometic device, can be used to interrogate the effluent from the channel structure. The spectral signature of the effluent over time tends to change as the characteristics of the banded materials change. This spectral signature can be used to monitor and identify moieties separated using these methods.

Moieties that are reversibly bound can be eluted from the channel structure using appropriate eluents, such as buffers of high ionic strength or of high surface activity, or a combination thereof. The eluted moieties can also be detected using appropriate spectrophotometic devices and methods. Moieties that are covalently linked to the surface of a channel structure can be eluted by cleaving the link between the channel structure and the immobilized moiety. For example, certain bonds are photolable while other can be broken using relatively gentle chemical reactions or enzymatic activities. When the covalent linkage involves biological moieties, enzymatic activities or chemical activities can be used to break these bonds.

General methods of sample preparation, injection and detection for microchips are available in the art (He et al., Anal. Chem. 70:3790-3797 (1998); Kutter et al., Anal. Chem. 70:3291-3297 (1998); and Figeys et al., Anal. Chem. 300A (May 2000)) and are generally applicable to the present invention.

IV. A Method of Performing a Bioassay, a Biochemical Reaction or a Chemical Reaction Using a Platform

A fourth aspect of the present invention is a method of performing a biochemical reaction, a bioassay or a chemical reaction that includes: providing a platform of the present invention, providing one or more reagents for use in the chemical reaction, biochemical reaction or bioassay, contacting the platform with the reagents, moving the reagents through channels on the platform such that the reagents are contacted and a assay is performed and optionally detecting at least one reactant or product of the bioassay. In one aspect of the present invention includes a method for performing chemical reactions or biochemical reactions that includes: providing a platform of the present invention, providing one or more reagents for use in the reactions, contacting the platform with the reagents, moving and optionally mixing the reagents through channels on the platform such that reactions can occur and optionally detecting the occurrence of a chemical reaction or a biochemical reaction.

a. Bioassay

In this aspect of the present invention, any appropriately configured microchip of the present invention can be utilized. Preferably, the biochip includes particles in the coating film, but that is not a requirement. Furthermore, the biochip preferably includes chemical groups or biological groups, preferably exposed to the channel structures that can include wells or chambers. The channel structure is preferably relatively long in length such that chemical moieties or biological moieties present in a sample can interact with the chemical groups or biological groups exposed on the surface of the channel structures. In this aspect of the present invention, chemical moieties or biological moieties in a sample can interact with chemical groups or biological groups on the channel structures. Preferably, the interactions between the moiety and group is specific in nature and are preferably characterized as short-range interactions. For example, preferred interactions are specific binding interactions including receptor-ligand, antibody-antigen, nucleic acid-nucleic acid, nucleic acid-protein, protein-protein or the like. In one aspect of the present invention, spectrophotometic devices can be used to determine light scattering, light absorption, light emission or other light based detection phenomenon to determine the bioassay results. Different moieties have different spectral signatures, such as absorption of light of various wavelengths. Fluorescent moieties exhibit excitation spectra and emission spectra that can be interrogated and detected. Particulates exhibit light scattering profiles as well. In addition, chromogenic moieties have spectral signatures that can be detected. Also, moieties can be labeled with detectable labels, such as chromogens, particulates, fluorophores or radioisotopes or radioprobes to facilitate detecting these moieties in the channel structure. In another aspect of the present invention, a third reagent can be used to detect specific binding. For example, a detectably labeled antibody can be used as a reagent to bind with and thus detect the localization or presence of a specific binding reaction.

In one non-limiting aspect of the present invention, binding assays, such as immunoassays, nucleic acid hybridization assays and receptor-based assays can be performed using a platform of the present invention. Binding molecules can be bound on the channel surface and target molecules can be introduced into the channel structure, or vis a vis. Through the use of labeled binding molecules or target molecules, the recognition events between binding molecules and target molecules can be detectable by a detectable label or detectable signal, such as a particle or enzymatic activity. The binding molecule can include, but are not limited to antibodies, nucleic acid molecules such as single stranded or double stranded DNA or RNA or combinations thereof, molecular receptors or active fragments thereof. The binding molecules can include but are not limited to nucleic acid molecules such as single stranded or double stranded DNA or RNA or combinations thereof, enzymes, peptides, proteins, polymers, ions, metal ions and low molecular weight organic species such as toxins, drugs, pharmaceuticals, illicit drugs, explosive, environmental toxins and the like.

Preferred sample preparation methods include standard laboratory sample preparation methods, such as those routinely applicable to the clinical or biological or biomedical research laboratory. For example, standard sample preparation methods including separation methods including centrifugation, filtration or chromatography. Also, treatment of samples with reagents to prepare them for particular assays, such as cell lysis, selective cell lysis, proteases, lipases or other enzyme treatments can be utilized. Other sample preparation methods, such as maintaining appropriate temperature, heat inactivation of enzymes such as proteases using heat, inhibitors or chelators, freeze drying of samples, freeze thaw procedures or mechanical treatment of samples such as with a French Press can be utilized. In the alternative, biochips can be used for sample preparation. In particular, cell sample processing biochips can be used to isolate, manipulate or purify cell populations from a sample, in particular blood samples or other fluid samples. For example, cell lysis buffers can be used to reduce the number or percentage of red blood cells in a sample (see for example U.S. application Ser. No. 09/686,737 herein incorporated by reference in its entirety). In addition, multi-force biochips and methods of using same can be used to prepare samples for use in assays using biochips (see for example U.S. application Ser. No. 60/239,299 herein incorporated by reference in its entirety).

Sample injection methods and devices appropriate for injecting a sample are available in the art and can be used in the present invention. Preferred sample injection devices are microfluidic in nature such that the volume and pressure of the output can be modulated as needed. For example, piezo dispensation devices can be utilized to dispense fluids onto or into a biochip. In the alternative, solonoid devices can be used. Furthermore, syringe based systems, such as commercially available from Hamilton, can be utilized. Several systems are available for microfluidic manipulations, such as are provided in the high throughput screening (including drug screening) as well as micromanufacture and electronic chip manufacture industries. These dispensation devices can engage a biochip or a channel thereon using methods known in the art and available structures.

Sample detection devices for use in the present invention are preferably optical in nature, but that need not be the case. Detection means can be positioned at a locus where test results are available, such as on a biochip, within a biochip or an effluent from a biochip. These detection means can be, for example, light detection devices such as CCD's or the like that can detect light scattering, absorption of light or light emission from a locus, such as fluorescence or chemiluminescense. In addition, commercially available liquid dispenser systems such as those offered by Packard Scientific, Inc. and Hamilton, Inc. and colorimetric scanner systems such as those offered by Techan, Inc. and Bio-Rad Inc. for 96 and 384 well titer plates can be used for sample preparation and detection in their original forms or modified forms.

b. Chemical Reactions or Biochemical Reactions

In addition to binding reactions, biochips of the present invention can be utilized to perform chemical reactions or biochemical reactions. Chemical reactions, such as synthesis or detection of moieties of interest using chemical reactions, can take place on or within a biochip of the present invention.

1. Chemical Reactions

A variety of chemical reactions can take place on or within a biochip of the present invention. For example, the channel present on biochips of the present invention can be utilized to sequentially add a moiety of interest to a chemical reaction, particularly when the chemical reaction is taking place at least in part on an solid support, such as the channel itself or particles imbedded therein. This aspect of the present invention is particularly useful for chemical synthesis involving sequential addition of moieties, such as the synthesis of polymers. Generally, any solid phase chemical synthesis method can be adapted for use in a biochip of the present invention. Examples of a preferred synthesis include the synthesis of non-naturally occurring polymers such as protein nucleic acid molecules, or “PNAs.” When the polymer is completed, the polymer can be cleaved from its solid support using chemistry appropriate for the synthetic product.

In the alternative, a chemical reaction can take place on or within a biochip of the present invention, particularly in the channels thereof, where mixing and detection of reaction products can take place. In one preferred aspect of the present invention, the channel structures of the present invention can separate moieties in a mixture, such as a sample, the separated moieties can sequentially be added to a reaction area, such as another channel, the same channel or a reactions chamber. In the reaction area, the moiety and reactants are contacted with each other. If a moiety that reacts with a reactant are mixed together, then a product can be formed. This product can preferably be detected using a detection structure. In one preferred aspect of the present invention, a color or chromogen or fluorophore is activated or modulated upon such a reaction. For example, soluble iron ions mixed with base will form a precipitate brown in color. This precipitate can be detected using light scattering, light reflectance or detection of color in the sample. This test can be used, for example, to determine iron ion concentrations in a sample, such as blood as a measure of hemoglobin or anemia or in a water sample as a measure of water hardness. Other chemical reactions that are routinely used in clinical or research settings can be adapted to the present invention. Such reactions can be simple, such as in the iron example, or complex. Similarly, the present invention may be used to make modifications or derivatives of compounds such as by adding or removing chemical groups from moieties on particles. Therefore libraries of compounds may be generated using combinatorial chemistry techniques (see, generally, for example “Comprehensive survey of combinatorial library synthesis, 1999” by Dolle, Journal of Combinatorial Chemistry 2:383-433 (2000).

2. Biochemical Reactions

In addition to chemical reactions, biochemical reactions can be performed on a biochip of the present invention. For example, synthetic peptides or nucleic acid molecules can be synthesized using biochips of the present invention. As discussed above, the biochips of the present invention are particularly useful for chemical synthesis utilizing sequential addition of reactant. In a preferred aspect of the present invention, a chemical or biochemical reaction, such as the formation of peptide bonds or phoshodiester bonds can take place on a solid phase, such as a locus on a biochip, such as a channel. Using chemistry available in the art, such as solid phase synthesis of nucleic acids or polypeptides, appropriate reactants can be passed along a channel such that sequential monomers are added to a growing polymer. When the polymer is complete, the polymer can be freed from the solid phase using an appropriate cleaving agent, such as an enzyme.

Like the chemical reactions discussed above, the separation or transfer of reactants can be used to contact reactants to so that biochemical reactions can take place. Preferably, when a biochemical reaction takes place, an appropriate readout, such as the generation of a chomogen, particulate or light, is generated. In one example, the moieties within a sample can be separated using a channel structure on a biochip of the present invention. If a moiety such as an enzyme having a particular activity is contacted with an appropriate substrate, then the enzyme can act on that substrate. If the substrate is chromogenic or a pre-chromogen, then the activity of the enzyme on the substrate can alter the optical characteristics of the reaction mixture. For example, beta-galactosidase can act upon a variety of substrates, such as MUG or ONPG. When glucuronidase acts upon MUG, a fluorogenic product is produced. When beta-galactosidase acts upon ONPG, a yellow chromogen results. These changes in fluorescence or color can be detected using appropriate detection devices, such as spectrophotometric devices.

This type of method is useful for detecting enzymes, but can also be used to detect coliforms or fecal coliforms in environmental samples. For example, an environmental sample or food sample, such as water or meat, can be processed to obtain and optionally concentrate enzymes therein. If the sample has glucuronidase, then the reaction will fluoresce and fecal coliforms are likely present in the sample. If the sample has beta-galactosidase, then the reaction will turn yellow and coliforms are likely present in the sample. Similar types of chromogenic, fluorogenic or precipitating enzymatic substrates are available for a wide variety enzymes. Many of these enzymes have diagnostic value for diseases or conditions of subjects, such as animals or humans, or for the detection of etiological agents in samples

V. A Method of Performing Cell Separation or Cell Capture

A fifth aspect of the present invention is a method of performing separation or capture of particles, cells, or components thereof. The method includes providing a platform of the present invention, introducing a sample having cells into at least on channel structure on the platform such as by injection, moving the sample or at least one component thereof through the at least one channel structure on the platform such that the cells within the sample are captured or separated, and optionally detecting the cells, activity, or a component thereof. Cells may be separated according to their physical properties and may be captured by cellular binding or cellular interactions.

Cell separation may be performed utilizing a variety of techniques known to those skilled in the art such as those that separate cells by their isolectric point (PI), size, density, granularity, dielectric constant, or a combination of these. Imbedded particles may assist in the separation of cells by altering features of the channel. For example, imbedded particles may change the environmental pH, hydrophobicity, size, or charge of the channel allowing selection of target cells. Separation may be by positive separation such that the target cell is allowed to pass through a barrier or by negative selection such that the target cell is prevented from crossing a barrier. One skilled in the art would recognize that the method of separation may be different depending on multiple factors such as, but not limited to, the physical characteristics of the target cell and availability of reagents.

Captures may be performed utilizing a variety of techniques known to those skilled in the art such as those that capture by covalent binding, ionic binding, or vanderwaals forces. For example, antibodies are commonly used in assays to specifically bind a target cell in techniques such as ELISA and immuno-precipitation and are useable with the present invention. Particles may be imbedded and modified with bioactive molecules such that the particles may capture a target population or a non-target population. Preferably cell capture is performed by specifically binding the biomolecules on the surface of an imbedded particle to a target cell's surface receptor. One skilled in the art would recognize the method of capture may be different depending on multiple factors such as, but not limited to, the chemical characteristics of the target cell and the availability of reagents.

### EXAMPLES

### Example 1

Method of Manufacture of a Biochip

The following example refers to one aspect of a method of making a biochip of the present invention. This method includes making two polymer preparations and one particle preparation. These three preparations are mixed and provided as a coating film that is selectively polymerized using photomasking.

A first polymer preparation (Preparation A) is made by mixing 1:100 grams of poly(acrylamide/acrylic acid) (90:10, sodium salt, MW 200,000 daltons) and 20 grams of N-(3-aminopropyl)methacrylamide hydrochloride (both from Polysciences) in 300 mL of 0.1 phosphate buffer (pH 7.4) under stirring while under nitrogen. Twenty grams of 1-ethyl-3-(3-dimethylaminopropyl)carbodiimide hydrochloride (EDC) are added and are stirred continuously for about eight hours. The reaction mixture is filtered and the resulting solution dialyzed against cellulose acetate membrane (MW cut off of 3,000 daltons). The modified polymer is precipitated from methanol.

A second polymer preparation (Preparation B) is made by providing an oligoDNA with terminal amino groups (from MWG Biotech) and mixing with an equal amount of bis(sulfosuccinimidyl)suberate (from Pierce) in 50 millimolar phosphate buffer (pH 8.5 in 1 millimolar EDTA). After three hours, an equal amount of N-(3-aminopropyl)methacrylamide hydrochloride is added to the mixture and the resulting mixture stirred for three hours.

A particle preparation (Preparation C) is made by providing 50 grams of quarts particles (from Duke Scientific, 0.35-3.5 micrometer) in a three necked flask with a thermometer, a stirrer, a dropping funnel and a reflux condenser. The mixture is charged with 150 millileters of toluene, 50 milliliters of pyridine, 10 grams of vinyltrichlorosilane and 20 grams of n-octadecyltrichlorosilane (both from Gelest). This mixture is refluxed for about ten hours. The particles are prepared by washing with toluene, then acetone and then methanol.

A preparation of surface coating film is made be preparing a viscous solution. This solution includes a mixture of Preparation A (10%), Preparation B (10%), Preparation C (20%), poly(ethylene glycol) dimethacrylate (n+600) (40%) (from Polyscience), 2,2-dimethoxy-2-phenylacetophenone (2%) and water (18%). This solution is spin-coated onto a substrate of glass using spin-coating or spin-casting methods known in the art to provide a layer of polymerizable composition on the substrate. The polymerizable composition has a thickness of about 100 nanometers. The polymerizable composition with the substrate is baked at about 95C for about one hour under nitrogen.

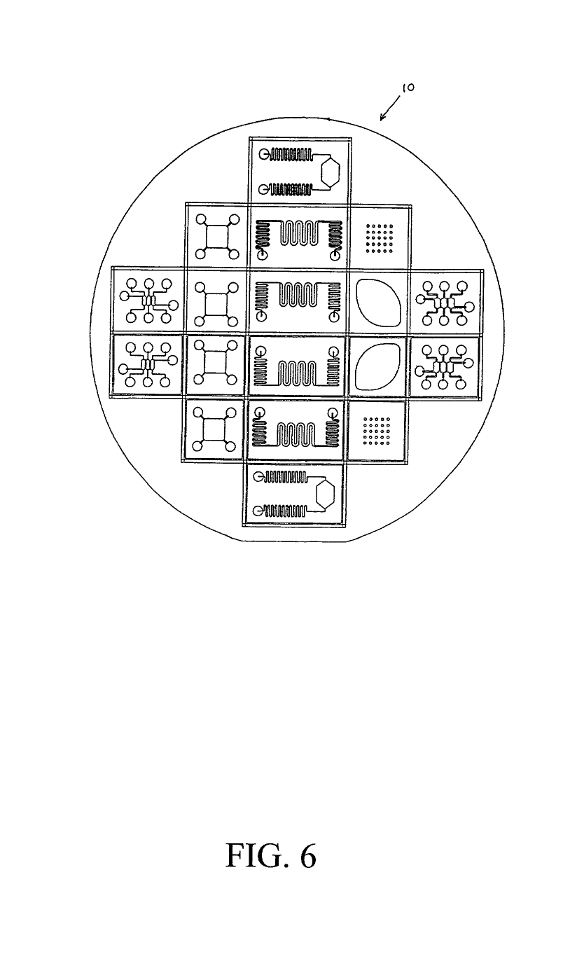

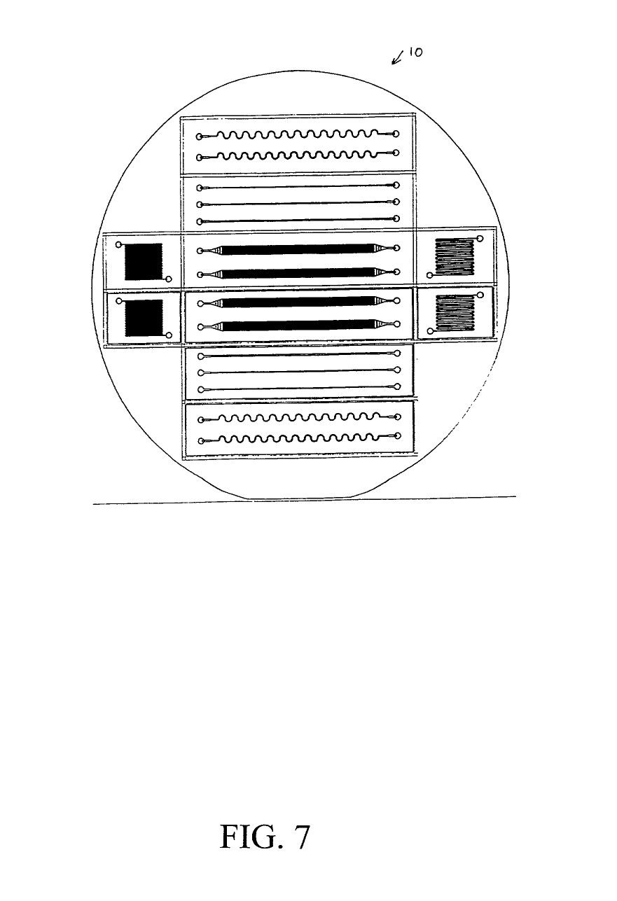

Selective photocuring of the baked coating film on the substrate is accomplished using UV light (about 350 nm to about 380 nm) through a photomask for about fifteen minutes. The photomask provides UV blocking regions that provide a pattern of channel structures as shown in FIG. 6 and FIG. 7. The channel structures have dimensions of about 100 micrometers ×about 10 micrometers. The resulting polymerized layer on the substrate is then baked at about 95° C. for about one hour under nitrogen. The baked structure is then washed with water and methanol to expose the channel structures on the coating film that were encoded in the UV blocking regions of the photomask. The channel structures can optionally take the form of wells or chambers. Optionally, a covering structure made of thin glass or PDMS is place on top of the resulting coating film to form channel structures that are at least partially closed.

In one method, SU-8 (Microchem, MA), a negative photoresist that can produce relatively thick films, is used. Epoxy-group containing silane treated silica or quartz particles having an average diameter of between about 3 micrometers and about 5 micrometers are added to a SU-8 solution. This solution with particles is spin coated onto the surface of a subtrate and the substrate with spin cast solution is baked at about 90° C. for about 30 minutes. This baked substrate is then radiated with UV light using a photomask designed to code for channel structures. After this photomasking, the substrate is baked again at about 90° C. for about 30 minutes. In order to remove uncured parts of the coating film, microchannels are exposed by washing the structure. One the surface of the channels, particles are exposed to the channel structure. These particles can be chemically or biochemically modified to obtain desired surface chemistry for separation or analytical purposes. This platform is optionally covered with a thin glass plate or plastic film, optionally with an adhesive on the side that contacts the platform. This glass plate or plastic film forms closed channels. The resulting platform can then be operably linked to fluid dispensation devices, fluid control devices, electric power supplies and the like. These chips can preferably be used for separation methods, including HPLC, CE or CEC.

### Example 2

Method of Manufacture of a Biochip on a Silicon Wafer or by Molding

SU-8 negative epoxy-based photoresist (Microchem, MA) is spin coated onto a silicon wafer. The temperature of the wafer is raised slowly from about room temperature to about 95° C. and baked at about 95° C. for about two hours. The wafer is then exposed under UV light (about 350 nm) with photomask from about five to about twenty minutes. The wafer is then baked again at about 95° C. for about one hour. The wafer is developed in SU-8 developing solvent.

Silica particles having a diameter of about five micrometers are added to a mixture of one hundred fifty milliliters toluene, fifty milliliters pyridine, and ten grams vinyldimethylcholorosilane. The mixture containing silica particles is then refluxed for about ten hours and washed with toluene, then acetone, then methanol.

SYLGARD 184 silicone elasotomer (Dow Coming) is mixed with its supplied base and curing agent in a ratio of ten parts base to one part curing agent by weight. The silica mixture is added to the SYLGARD mixture, mixed well, and degassed with a vacuum pump for about five minutes. This mixture is poured on the SU-8 molding device and heated in an oven at about 70° C. for about six hours. The imbedded silicon device is peeled off the SU-8 molding after cooling. Further surface modifications and bonding with a glass or plastic cover may be performed with the prepared device.

### Example 3

Cell Capturing and Patterning Using a Particle Imbedded Chip

To demonstrate the capture and separation of cell, fetal cells were captured from maternal blood. However, other cells may be captured using the same technique with different antibody and slight modification that will be apparent to one skilled in the art.

A biochip device prepared by polymerization of surface coating film with imbedded particles, as described in Example 2, was reacted with aminopropyltrimethoxysilane in toluene, and then with glutaric dialdehyde. Streptavidin (Pierce) are bonded on the biochip surface through the aldehyde group.

The maternal blood was prepared by centrifugation at 1500 rpm, and then incubated with biotinated anti-Human CD 71 antibody ( Leinco Technologies, MO) in PBS buffer (containing 0.5% BSA) for 15 minutes. This sample was then applied onto the biochip device, the fetal cells were captured on the surface of the biochip due to biotin-streptavidin interaction. The biochip was washed with PBS buffer and dried by air purge.

The cells captured on the biochips were stained for hemoglobin by Benzidine-Wright-Giemsa procedure. The air dried biochip was immersed in methanol for 5 minutes, 1% benzidine for 1.5 minutes, 50% ethanol/peroxide solution for 1.5 minutes, DI water for 5 seconds, Wright-Giemsa solution (Sigma) for 10 minutes, and water for 10 seconds. The biochips were air dried. The cells thus stained can be observed under a typical microscope.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified

what is claimed is:

1. A platform, comprising:
a) a surface;
b) a coating film;
c) a channel structure;
wherein said coating film defines in part said channel structure;
wherein said platform comprises a microchip;
wherein said coating film comprises a particle.

2. The platform of claim 1, wherein said surface comprises at least in part silica, glass, quartz, fused silica, polymer, plastic, metal, metal oxide, PTFE, polysilicon, silicon nitride, ceramic, composit or carbon.

3. The platform of claim 1, wherein said surface comprises a magnetic element, an electromagnetic element, an acoustic element or a dielectric element.

4. The platform of claim 1, wherein said surface is between about 10 micrometers and about 20 centimeters in length or width.

5. The platform of claim 1, wherein said surface is between about 0.1 micrometers and about 10 centimeters in thickness.

6. The platform of claim 1, wherein said coating film comprises a polymer, homopolymer, copolymer, cross-linked polymer, partially polymerized polymer or a cross-linked polymer network.

7. The platform of claim 1, wherein said coating film comprises a hydrophobic polymer or a hydrophilic polymer.

8. The platform of claim 1, wherein said coating film comprises at least in part polyethyleneglycol, polyurethanes, polyacrylates, polyacrylamides, polymethylacrylamide, polyvinyl alcohol, polyvinylpyrrolidone, polyamino acids, polysaccharides and polysiloxanes.

9. The platform of claim 1, wherein said coating film is biocompatable.

10. The platform of claim 1, wherein said coating film is between about 10 micrometers and about 20 centimeters in length or width.

11. The platform of claim 1, wherein said coating film is between about 0.1 micrometers and about 10 millimeters in thickness.

12. The platform of claim 1, wherein said coating film comprises at least in part a biological group.

13. The platform of claim 12, wherein said biological group comprises at least in part a biomolecule, polypeptide, antibody, receptor, protein, nucleic acid, small molecule, carbohydrate, lipid or combinations thereof.

14. The platform of claim 12, wherein said biological group interacts with a biological moiety or chemical moiety by electrostatic interactions, ionic interactions, hydrogen bonding or hydrophobic interactions.

15. The platform of claim 12, wherein said biological group interacts with a biological moiety by nucleic acid—nucleic acid interactions, nucleic acid—protein interactions, antigen—antibody interactions, receptor—ligand interactions or protein—small molecule interactions.

16. The platform of claim 12, wherein said biological group is present substantially throughout said coating film or on the surface of said coating film.

17. The platform of claim 1, wherein said coating film comprises at least in part a chemical group.

18. The platform of claim 17, wherein said chemical group comprises at least in part an alkyl group, a charged group, a positively charged group, a negatively charged group, small molecules or combinations thereof.

19. The platform of claim 17, wherein said chemical group interacts with a chemical moiety or biological moiety by electrostatic interactions, ionic interactions, hydrogen bonding, hydrophobic interactions or covalent linking.

20. The platform of claim 17, wherein said chemical group is present substantially throughout said coating film or on the surface of said coating film.

21. The platform of claim 1, wherein said particle is imbedded within said coating film.

22. The platform of claim 21, wherein said particles comprise between about 0.1% and about 99.9% volume/volume of said polymer coating.

23. The platform of claim 21, wherein said particles comprise at least in part glass, silica, quartz, fused silica, polymer, metal oxide, polystyrene, PMMA, plastic, polysaccharides or polyimide.

24. The platform of claim 21, wherein said particle size, on average, is between about 0.05 micrometers and about 500 micrometers.

25. The platform of claim 21, wherein said particles are biocompatable.

26. The platform of claim 21, wherein said particles comprises at least in part a biological group.

27. The platform of claim 21, wherein said particles comprises at least in part a chemical group.

28. The platform of claim 1, wherein said channel structure comprises open channels or closed channels.

29. The platform of claim 1, wherein at least a portion of said channel structure is defined by said surface or a covering structure.

30. The platform of claim 1, wherein at least a portion of said channel structure is defined by said coating film.

31. The platform of claim 1, wherein at least a portion of said channel structure is defined by selective polymerization of said coating film.

32. The platform of claim 1, wherein said channel structures forms at least one island.

33. The platform of claim 1, wherein said channel structure has a shape in cross section that is substantially square, oval, crescent, half-circle or rectangular.

34. The platform of claim 1, wherein said channel structure is linear, circular, coiled, curved, saw-toothed or switchback along at least a portion of its length.

35. The platform of claim 1, further comprising a magnetic element, an electromagnetic element, an acoustic element or a dielectric element.

36. A method of making a platform that comprises at least one channel structure, comprising:
a) providing a surface;
b) contacting said surface with a polymerizable composition comprising:
1. unpolymerized polymer subunits;
2. at least one polymerization initiator;
c) selectively polymerizing said polymerizable composition at loci to form a platform that comprises a polymerized layer that defines at least in part at least one channel structure.

37. The method of claim 36, wherein said surface comprises at least in part silica, glass, quartz, fused silica, polymer, plastic, metal, metal oxide, PTFE, polysilicon, silicon nitride, ceramic, composit or carbon.

38. The method of claim 36, wherein said surface comprises a magnetic element, an electromagnetic element, an acoustic element or a dielectric element.

39. The method of claim 36, wherein said unpolymerized polymer subunits comprise monomers, macromonomers or combinations thereof.

40. The method of claim 36, wherein said unpolymerized polymer subunits comprise partially polymerized polymer.

41. The method of claim 36, wherein said unpolymerized polymer subunits polymerize to form a homopolymer, copolymer, cross-linked polymer or a cross-linked polymer network

42. The method of claim 36, wherein said unpolymerized polymer subunits polymerize to form a hydrophobic polymer or a hydrophilic polymer.

43. The method of claim 36, wherein said unpolymerized polymer subunits are biocompatable.

44. The method of claim 36, wherein said unpolymerized polymer subunits comprise subunits of at least one polymer selected from the group consisting of acrylic, methacrylic, vinylbenzyl, vinyl, epoxy, polymers comprising pendant alpha,beta unsaturated ketones, polymers comprising pendant chalione moieties and polymers comprising cinnamates.

45. The method of claim 36, wherein said polymerization initiator comprises a photointiator or a thermal initiator.

46. The method of claim 38, wherein said photoinitiator is selected from the group consisting of 2,2-dimethoxy-2-phenyl acetophenone, benzophenone, anthraquinone, diethoxyacetophenone, p-dimethylaminoacetophenone, mono-acylphosphineoxides, bis-acylphosphineoxides, bis(2,4,6-trimethylbenzoly)-phenylphosphine oxide and 1-[4-(2-hydroxyethoxy)-phenyl]-2-hydroxy-2-methyl-1-propane-1-one.

47. The method of claim 36, wherein said polymerization initiator is provided at a concentration of between about 0.1% and about 10% weight/volume.

48. The method of claim 36, wherein said selectively polymerizing comprises localized initiation of said at least one polymerization initiator.

49. The method of claim 48, wherein said localized initiation comprises localizing electromagnetic radiation, UV light or laser light.

50. The method of claim 49, wherein said electromagnetic radiation is of a wavelength between about 180 nanometers and about 600 nanometers.

51. The method of claim 49, wherein said electromagnetic radiation is localized using a masking.

52. The method of claim 51, wherein said masking comprises a photomask, printed transparancy film, transparent areas or windows.

53. The method of claim 36, wherein said polymerizable composition comprises at least in part a biological group.

54. The method of claim 53, wherein said biological group is present substantially throughout said coating film or on the surface of said coating film.

55. The method of claim 36, wherein said polymerizable composition comprises at least in part a chemical group.

56. The method of claim 55, wherein said chemical group is present substantially throughout said coating film or on the surface of said coating film.

57. The method of claim 36, wherein said polymerizable composition comprises a polymerizing functional group.

58. The method of claim 57, wherein said polymerizing functional group can form a bond with a polymer, a monomer or a particle.

59. The method of claim 57, wherein said polymerizing functional group is selected from the group consisting of acrylics, methacrylics, vinylbenzyls, vinyls, epoxies, alpha/beta usaturated ketones, cinnamates, chalione groups.

60. The method of claim 36, wherein said polymerized layer comprises a biological group or a chemical group.

61. The method of claim 60, wherein said biological groups or chemical groups result from selective polymerization.

62. The method of claim 60, wherein said biological groups or chemical groups result from modification of at least a portion of said polymerized layer.

63. The method of claim 60, wherein said biological groups or chemical groups result from coating of at least a portion of said polymerized layer.

64. The method of claim 60, wherein said polymerized layer comprises a polymer selected from the group consisting of polyethyleneglycol, polyurethanes, polyacrylates, polyacrylamides, polymethylacrylamide, polyvinyl alcohol, polyvinyl prolidone, polyamino acids, polysaccharides and polysiloxanes.

65. The method of claim 60, wherein said biological groups or said chemical groups are throughout the polymerized layer on the surface of aid polymerized layer.

66. The method of claim 36, wherein said polymerizable composition or coating film further comprises particles.

67. The method of claim 66, wherein said particles comprise between about 0.1% and about 99.9% volume/volume of said coating film.

68. The method of claim 66, wherein said particles comprise at least in part glass, silica, quartz, fused silica, polymer, metal oxide, polystyrene, PMMA, plastic, polysaccharide or polyamide.

69. The method of claim 66, wherein said particle size, on average, is between about 0.1 micrometer and about 50 micrometers.

70. The method of claim 66, wherein said particles are biocompatable.

71. The method of claim 66, wherein said particles comprises at least in part a biological group.

72. The platform of claim 36, wherein said particles comprises at least in part a chemical group.

73. The method of claim 66, wherein said particles comprise a polymerizing functional group.

74. The method of claim 73, wherein said polymerizing functional group can form a bond with a polymer, a monomer or another particle.

75. The method of claim 73, wherein said polymerizing functional group is selected from the group consisting of acrylics, methacrylics, vinylbenzyls, vinyls, epoxies, alpha/beta usaturated ketones, cinnamates, chalione groups.

76. The method of claim 36, wherein said at least one channel structure comprises open channels or closed channels, wells or chambers.

77. The method of claim 36, wherein at least a portion of said at least one channel structure is defined by said surface or covering structure.

78. The method of claim 36, wherein at least a portion of said channel structure is defined by said coating film

79. The method of claim 36, wherein said at least one channel structures forms at least one island.

80. The method of claim 36, wherein said at least one channel structure has a shape in cross section that is substantially square, oval, crescent, half-circle or rectangular.

81. The method of claim 36, wherein said at least one channel structure is linear, circular, coiled, curved, saw-toothed or switchback along at least a portion of its length.

82. The method of claim 36, wherein said channel structures are formed by removing unpolymerized or partially polymerized materials.

83. The method of claim 82, wherein said removing is by washing.

84. The method of claim 36, wherein said platform further comprising a magnetic element, an electromagnetic element, an acoustic element or a dielectric element.

85. A platform made by the method of claim 36.

86. The platform of claim 85, wherein said platform defines a chip or a microchip.

87. A method of separating moieties, comprising:
a) providing a platform of claim 1;
b) providing a sample containing moieties;
c) contacting said platform with said sample;
d) moving said sample through channels on said platform such that moieties within said sample are separated; and
e) optionally detected at least one moiety.

88. A method of performing a bioassay, comprising:
a) providing a platform of claim 1;
b) providing one or more reagents for use in said bioassay;
c) contacting said platform with said reagents;
d) moving said reagents through channels on said platform such that said reagents are contacted and a bioassay is performed; and
e) optionally detecting at least one reactant or product of said bioassay.

89. A method of performing a chemical reaction, comprising:
a) providing a platform of claim 1;
b) providing one or more reagents for use in said chemical reaction;
c) contacting said platform with said reagents;
d) moving said reagents through channels on said platform such that said reagents are contacted and a chemical reaction is performed; and
e) optionally detecting at least one reactant or product of said chemical reaction.

90. A method of performing high performance liquid chromatography; comprising:
a) providing a platform of claim 1;
b) injecting a sample into at least one channel structure on said platform;
c) performing high performance liquid chromatography using said at least one channel structure; and
d) optionally detecting a moiety separated by said high performance liquid chromatography.

91. A method for performing capillary electrophoresis, comprising:
a) providing a platform of claim 1;
b) injecting a sample into at least one channel structure on said platform;
c) performing capillary electrophoresis using said at least one channel structure; and
d) optionally detecting a moiety separated by said capillary electrophoresis.

92. A method for performing capillary electrochromatography, comprising:
a) providing a platform of claim 1;
b) injecting a sample into at least one channel structure on said platform;
c) performing capillary electrochromatography using said at least one channel structure; and
d) optionally detecting a moiety separated by said capillary electrochromatography.

93. A method for cell separating comprising:
a) providing a platform of claim 1;
b) introducing a sample having cells into at least one channel structure on said platform;
c) moving said sample or at least one component thereof through said at least one channel structure on said platform such that said cells within said sample are separated; and
d) optionally detecting said cells.

94. A method for capturing a cell comprising:
a) providing a platform of claim 1;
b) introducing a sample having cells into at least one channel structure on said platform;
c) moving said sample or at least one component thereof through said at least one channel structure on said platform such that said cells within said sample are captured; and
d) optionally detecting said cells.
